SEQUENCE LISTING (24 sequences)

SEQ ID NO: 1 - DNA, 6586 bp, Homo Sapiens
  CDS (440)..(5386)
ctggggagccggcgctggaggtggtgagtggcgtggggactgtgtcgagggggtccccaaggtgccggaccctgcggaggggcgaagtttcggcactggggagggcgtgcggacgctttccctacaggcgaccactgctctgcgggcgggtggtcttagctccagtcccccattcagttcctcagcattccaggtcggcggcgaaggggtccccgaacgaagggcgcaaggcagcgtctctgctgggaccgggaagccggacttcagggcctctcggcccgtgggcttctccccgagtctccccgagtcggttggcattaagagtttagcagatactttcagaaatggatacataagaaatggctggaaatcaaatgaatgtccaaagaagagcttagggtcttagtaacattcttttttaaaataactgtctgccaaaatgtcattacacagtactcataatagaaataacagcggtgatattcttgatattccttcttcccaaaatagttcatcactgaatgccctcacccacagtagccgacttaagctgcatttgaagtcggatatgtcagaatgtgaaaatgatgatccattattgagatctgcaggtaaagtcagagacataaatagaacttatgttatttctgccagtagaaaaacagcagacatgccccttacccctaatcctgtaggtagattggcacttcagaggagaactacaaggaacaaagaatcatctttgcttgttagtgagttggaagacacaactgaaaaaacagcagaaacacgtcttacattacaacgtcgtgctaaaacagattctgcagaaaagtggaaaacagctgaaatagattctgtcaaaatgacactgaatgtgggaggtgaaacagaaaataatggtgtttctaaggaaagtagaacaaatgtaaggattgtaaataatgctaaaaactcttttgttgcctcttctgtacctttagatgaagatccacaggtcattgaaatgatggctgataagaaatacaaagaaacattttctgcccccagtagagcaaatgaaaatgttgcacttaagtactcaagtaatagaccacccattgcttccctgagtcagactgaagttgttagatcaggacacttgacaacgaaacctactcagagcaagttggatatcaaagtgttgggaacaggaaacttgtatcatagaagtattgggaaggaaattgcaaaaacttcaaataaatttgggagcttagaaaaaagaacacctacaaaatgtacaacagaacacaaactgacaacaaagtgcagcctgcctcagcttaagagcccagctccatcaatactgaagaatagaatgtctaaccttcaagttaaacaaagaccaaaaagttcctttcttgcaaataaacaggaaagatccgcagaaaatacaattcttcccgaagaagaaactgtagttcagaacacctctgcaggaaaagaccccttaaaagtagagaatagtcaagtgacagtggcagtacgcgtaagacctttcaccaagagagagaagattgaaaaagcatcccaggtagtcttcatgagtgggaaagaaataactgtggaacaccctgacacgaaacaagtttataattttatttatgatgtttcattctggtcttttgatgaatgtcatcctcactacgctagccagacaactgtctatgagaagctagcagcaccactcctagaaagagccttcgaaggcttcaatacctgtctttttgcttatggtcagactggctctggaaaatcatatacgatgatgggatttagtgaagaaccaggaataattccaagattttgtgaagatcttttttctcaagtagccagaaaacaaacccaagaggtcagctatcacattgaaatgagcttctttgaagtatataatgaaaaaattcacgaccttctggtttgtaaagatgaaaatgggcagagaaagcaaccactgagagtgagggaacatcctgtttatggaccatatgttgaagcactgtcaatgaacattgtcagttcttacgctgatatccagagttggctagaattgggaaataaacaaagagctactgctgctactggtatgaatgataaaagttcccgatctcattcagttttcaccctggtgatgacccagaccaagacagaatttgtggaaggggaagaacacgatcacagaataacaagtcgaattaacctaatagatctggcaggcagtgagcgctgctctacggctcacactaatggagatcgactaaaggaaggtgtgagtattaataagtccttgctaactttgggaaaagttatatctgcactttcggaacaagcaaaccaaaggagtgtttttattccttatcgtgaatctgttcttacatggctgttaaaagaaagtctgggtggaaattcaaaaactgcaatgattgctacgattagtcccgctgccagcaacatagaagaaacattaagcacacttagatatgctaaccaagcccgtttaatagtcaacattgctaaagtaaatgaagatatgaacgctaagttaattagagaattgaaggcagaaattgcaaagctaaaagctgctcagagaaacagtcggaatattgaccctgaacgatacaggctctgtcggcaagaaataacatccttaagaatgaaactgcatcaacaggagagagacatggcagaaatgcaaagagtgtggaaagaaaagtttgaacaagctgaaaaaagaaaacttcaagaaacaaaagagttacagaaagcaggaattatgtttcaaatggacaatcatttaccaaaccttgttaatctgaatgaagatccacaactatctgagatgctgctatatatgataaaagaaggaacaactacagttggaaagtataaaccaaactcaagccatgatattcagttatctggggtgctgattgctgatgatcattgtactatcaaaaattttggtgggacagtgagtattatcccagttggggaagcaaagacatatgtaaatggaaaacatattttggaaatcacagtattacgtcatggtgatcgagtgattcttggtggagatcattattttagatttaatcatccagtagaagtccagaaaggaaaaaggccatctggaagagatactcctataagtgagggtccaaaagactttgaatttgcaaaaaatgagttgctcatggcacagagatcacaacttgaagcagaaataaaagaggctcagttgaaggcaaaggaagaaatgatgcaaggaatccagattgcaaaagaaatggctcagcaagagctttcttctcaaaaagctgcatatgaaagcaaaataaaagcactggaagcagaactgagagaagagtctcaaaggaaaaaaatgcaggaaataaataaccagaaggctaatcacaaaattgaggaattagaaaaggcaaagcagcatcttgaacaggaaatatatgtcaacaaaaagcgattagaaatggagacattggctacaaaacaggctttagaagaccatagcatccgccatgcaagaattctggaagctttagaaactgaaaagcaaaaaattgctaaagaagtacaaattctacagcagaatcggaataatagggataaaacttttacagtgcagacaacttggagctctatgaaactctcaatgatgattcaggaagccaatgctatcagcagcaaattgaaaacatactatgtttttggcagacatgatatatcagataaaagtagttctgacacttctattcgggttcgtaacctgaaactaggaatctcaacattctggagtctggaaaagtttgaatctaaacttgcagcaatgaaagaactttatgagagtaatggtagtaacaggggtgaagatgccttttgtgatcctgaagatgaatgggaacccgacattacagatgcaccagtttcttcactttctagaaggaggagtaggagtttgatgaagaacagaagaatttctggttgtttacatgacatacaagtccatccaattaagaatttgcattcttcacattcatcaggtttaatggacaaatcaagcactatttactcaaattcagcagagtcctttcttcctggaatttgcaaagaattgattggttcttcgttagatttttttggacagagttatgatgaagaaagaactatagcagacagcctaattaatagttttcttaaaatttataatgggctatttgccatttccaaggctcatgaagaacaagatgaagaaagtcaagataacttgttttcttctgatcgagcaatccagtcacttactattcagactgcatgtgcttttgagcagctagtagtgctaatgaaacactggctgagtgatttactgccttgtaccaacatagcaagacttgaggatgagttgagacaagaagttaaaaaactgggaggctacttacagttatttttgcagggatgctgtttggatatttcatcaatgataaaagaggctcaaaagaatgcaatccaaattgtacaacaagctgtaaagtatgtggggcagttagcagttctgaaagggagcaagctacattttctagaaaacggtaacaataaagctgccagtgtccaggaggaattcatggatgctgtttgtgatggtgtaggcttaggaatgaagattttattagattctggactggaaaaagcaaaagaacttcagcatgaactctttaggcagtgtacaaaaaatgaggttaccaaagaaatgaaaactaatgccatgggattgattagatctcttgaaaacatctttgctgaatcgaaaattaaaagtttcagaaggcaagtacaagaagaaaactttgaataccaagatttcaagaggatggttaatcgtgctccagaattcttaaagttaaaacattgcttagagaaagctattgaaattattatttctgcactgaaaggatgccatagtgatataaatcttctccagacttgtgttgaaagtattcgcaacttggccagtgatttttacagtgacttcagtgtgccttctacttctgttggcagctatgagagtagagtaactcacattgtccaccaggaactagaatctctagctaagtctctcctcttttgttttgaatctgaagaaagccctgatttgttgaaaccctgggaaacttataatcaaaataccaaagaagaacaccaacaatctaaatcaagcgggattgacggcagtaagaataaaggtgtaccaaagcgtgtctatgagctccatggctcatccccagcagtgagctcagaggaatgcacacccagtaggattcagtgggtgtgaatactgatgtgtaggcacttttatgaccacccatgaaagaaaaagaacacttgctcggtaattttctttatgcaggagagtttaagagaaatcagcacagatatttcaaaaaagtccatgtctttttatctttaaaatatctatttatcaaaggccagacacagtggctcacgcctgtaatcccagcactttgggaggcgggcagatcacaaggtcaggagtttgagaccggcctggccaacatggtgaaaccccgtctctactaaaaatacaaaaatttgctgggcatggtggcgcgtgcctgtaatcccagctactaggggggctgaggcaggaggatcgcttgaacctgagaggcagaggttgcagtgagccaagatcatgccactttactccagtctgagcaacagaacgagacttagtcaaaataaataaataaataagtaaataaataaataaataaaatatcttttatctttaaagtgtttaacattggtatactgtctgtagttggttcattagtcgtttataaagggttattttctcatgagtggaaacctgaacaatcagttacctttgtgcctatgccttctctctcctcagacagctgggatgtttatggtgaaatggcctgtacaagtttaactaagacaacttaacttgcattgttaatcaaaaattcttttctcaaagggttaactggttgccattttgaatagtatgttcaagggtgtagcttcctgtttctttccaaattataagtagctacctaaatatagtataattatatattaataatatggcttgctggcacagtagtttaccctgttatctgtgtttcataatgggggctgtatgaatattatttaaaactaataaaatgttgccagaattatactaaactgttggatgagattaggagatcagaggctggaccttctcttgataatgcttgttttgttaaaggtataatgaaataatttgtatatgatttgatgaagattaaagacccttattttccacagctttaaaaaaaaacctttatttatgatcaagtaataaagataatattctacttgtgggatcttacattatggaaatagtttgacgtttttgacctcaagagtatgtataatttgaagagatactttgtaactatgcttgggtgatattgagcagttcctaaagaataattcattt

SEQ ID NO: 2 - protein, 1648 aa, Homo Sapiens
MSLHSTHNRNNSGDILDIPSSQNSSSLNALTHSSRLKLHLKSDMSECENDDPLLRSAGKVRDINRTYVISASRKTADMPLTPNPVGRLALQRRTTRNKESSLLVSELEDTTEKTAETRLTLQRRAKTDSAEKWKTAEIDSVKMTLNVGGETENNGVSKESRTNVRIVNNAKNSFVASSVPLDEDPQVIEMMADKKYKETFSAPSRANENVALKYSSNRPPIASLSQTEVVRSGHLTTKPTQSKLDIKVLGTGNLYHRSIGKEIAKTSNKFGSLEKRTPTKCTTEHKLTTKCSLPQLKSPAPSILKNRMSNLQVKQRPKSSFLANKQERSAENTILPEEETVVQNTSAGKDPLKVENSQVTVAVRVRPFTKREKIEKASQVVFMSGKEITVEHPDTKQVYNFIYDVSFWSFDECHPHYASQTTVYEKLAAPLLERAFEGFNTCLFAYGQTGSGKSYTMMGFSEEPGIIPRFCEDLFSQVARKQTQEVSYHIEMSFFEVYNEKIHDLLVCKDENGQRKQPLRVREHPVYGPYVEALSMNIVSSYADIQSWLELGNKQRATAATGMNDKSSRSHSVFTLVMTQTKTEFVEGEEHDHRITSRINLIDLAGSERCSTAHTNGDRLKEGVSINKSLLTLGKVISALSEQANQRSVFIPYRESVLTWLLKESLGGNSKTAMIATISPAASNIEETLSTLRYANQARLIVNIAKVNEDMNAKLIRELKAEIAKLKAAQRNSRNIDPERYRLCRQEITSLRMKLHQQERDMAEMQRVWKEKFEQAEKRKLQETKELQKAGIMFQMDNHLPNLVNLNEDPQLSEMLLYMIKEGTTTVGKYKPNSSHDIQLSGVLIADDHCTIKNFGGTVSIIPVGEAKTYVNGKHILEITVLRHGDRVILGGDHYFRFNHPVEVQKGKRPSGRDTPISEGPKDFEFAKNELLMAQRSQLEAEIKEAQLKAKEEMMQGIQIAKEMAQQELSSQKAAYESKIKALEAELREESQRKKMQEINNQKANHKIEELEKAKQHLEQEIYVNKKRLEMETLATKQALEDHSIRHARILEALETEKQKIAKEVQILQQNRNNRDKTFTVQTTWSSMKLSMMIQEANAISSKLKTYYVFGRHDISDKSSSDTSIRVRNLKLGISTFWSLEKFESKLAAMKELYESNGSNRGEDAFCDPEDEWEPDITDAPVSSLSRRRSRSLMKNRRISGCLHDIQVHPIKNLHSSHSSGLMDKSSTIYSNSAESFLPGICKELIGSSLDFFGQSYDEERTIADSLINSFLKIYNGLFAISKAHEEQDEESQDNLFSSDRAIQSLTIQTACAFEQLVVLMKHWLSDLLPCTNIARLEDELRQEVKKLGGYLQLFLQGCCLDISSMIKEAQKNAIQIVQQAVKYVGQLAVLKGSKLHFLENGNNKAASVQEEFMDAVCDGVGLGMKILLDSGLEKAKELQHELFRQCTKNEVTKEMKTNAMGLIRSLENIFAESKIKSFRRQVQEENFEYQDFKRMVNRAPEFLKLKHCLEKAIEIIISALKGCHSDINLLQTCVESIRNLASDFYSDFSVPSTSVGSYESRVTHIVHQELESLAKSLLFCFESEESPDLLKPWETYNQNTKEEHQQSKSSGIDGSKNKGVPKRVYELHGSSPAVSSEECTPSRIQWV

SEQ ID NO: 3 - protein, 354 aa, Homo Sapiens
NSQVTVAVRVRPFTKREKIEKASQVVFMSGKEITVEHPDTKQVYNFIYDVSFWSFDECHPHYASQTTVYEKLAAPLLERAFEGFNTCLFAYGQTGSGKSYTMMGFSEEPGIIPRFCEDLFSQVARKQTQEVSYHIEMSFFEVYNEKIHDLLVCKDENGQRKQPLRVREHPVYGPYVEALSMNIVSSYADIQSWLELGNKQRATAATGMNDKSSRSHSVFTLVMTQTKTEFVEGEEHDHRITSRINLIDLAGSERCSTAHTNGDRLKEGVSINKSLLTLGKVISALSEQANQRSVFIPYRESVLTWLLKESLGGNSKTAMIATISPAASNIEETLSTLRYANQARLIVNIAKVNE

SEQ ID NO: 4 - protein, 388 aa, Homo Sapiens
MVQNTSAGKDPLKVENSQVTVAVRVRPFTKREKIEKASQVVFMSGKEITVEHPDTKQVYNFIYDVSFWSFDECHPHYASQTTVYEKLAAPLLERAFEGFNTCLFAYGQTGSGKSYTMMGFSEEPGIIPRFCEDLFSQVARKQTQEVSYHIEMSFFEVYNEKIHDLLVCKDENGQRKQPLRVREHPVYGPYVEALSMNIVSSYADIQSWLELGNKQRATAATGMNDKSSRSHSVFTLVMTQTKTEFVEGEEHDHRITSRINLIDLAGSERCSTAHTNGDRLKEGVSINKSLLTLGKVISALSEQANQRSVFIPYRESVLTWLLKESLGGNSKTAMIATISPAASNIEETLSTLRYANQARLIVNIAKVNEDMNAKLIRELKLEHHHHHH

SEQ ID NO: 5 - protein, 378 aa, Homo Sapiens
MVQNTSAGKDPLKVENSQVTVAVRVRPFTKREKIEKASQVVFMSGKEITVEHPDTKQVYNFIYDVSFWSFDECHPHYASQTTVYEKLAAPLLERAFEGFNTCLFAYGQTGSGKSYTMMGFSEEPGIIPRFCEDLFSQVARKQTQEVSYHIEMSFFEVYNEKIHDLLVCKDENGQRKQPLRVREHPVYGPYVEALSMNIVSSYADIQSWLELGNKQRATAATGMNDKSSRSHSVFTLVMTQTKTEFVEGEEHDHRITSRINLIDLAGSERCSTAHTNGDRLKEGVSINKSLLTLGKVISALSEQANQRSVFIPYRESVLTWLLKESLGGNSKTAMIATISPAASNIEETLSTLRYANQARLIVNIAKVNEDLEHHHHHH

SEQ ID NO: 6 - protein, 376 aa, Homo Sapiens
MVENSQVTVAVRVRPFTKREKIEKASQVVFMSGKEITVEHPDTKQVYNFIYDVSFWSFDECHPHYASQTTVYEKLAAPLLERAFEGFNTCLFAYGQTGSGKSYTMMGFSEEPGIIPRFCEDLFSQVARKQTQEVSYHIEMSFFEVYNEKIHDLLVCKDENGQRKQPLRVREHPVYGPYVEALSMNIVSSYADIQSWLELGNKQRATAATGMNDKSSRSHSVFTLVMTQTKTEFVEGEEHDHRITSRINLIDLAGSERCSTAHTNGDRLKEGVSINKSLLTLGKVISALSEQANQRSVFIPYRESVLTWLLKESLGGNSKTAMIATISPAASNIEETLSTLRYANQARLIVNIAKVNEDMNAKLIRELKLEHHHHHH

SEQ ID NO: 7 - protein, 366 aa, Homo Sapiens
MVENSQVTVAVRVRPFTKREKIEKASQVVFMSGKEITVEHPDTKQVYNFIYDVSFWSFDECHPHYASQTTVYEKLAAPLLERAFEGFNTCLFAYGQTGSGKSYTMMGFSEEPGIIPRFCEDLFSQVARKQTQEVSYHIEMSFFEVYNEKIHDLLVCKDENGQRKQPLRVREHPVYGPYVEALSMNIVSSYADIQSWLELGNKQRATAATGMNDKSSRSHSVFTLVMTQTKTEFVEGEEHDHRITSRINLIDLAGSERCSTAHTNGDRLKEGVSINKSLLTLGKVISALSEQANQRSVFIPYRESVLTWLLKESLGGNSKTAMIATISPAASNIEETLSTLRYANQARLIVNIAKVNEDLEHHHHHH

SEQ ID NO: 8 - DNA, 21 bp, Artificial Sequence
  Synthetic
  misc_feature (1)..(21): double stranded
  misc_feature (20)..(21): Sequence is DNA/RNA hybrid wherein N = T
aaacugggaggcuacuuacnn

SEQ ID NO: 9 - DNA, 21 bp, Artificial Sequence
  Synthetic
  misc_feature (1)..(21): double stranded
  misc_feature (20)..(21): Sequence is DNA/RNA hybrid wherein N = T
cucacauuguccaccaggann

SEQ ID NO: 10 - DNA, 21 bp, Artificial Sequence
  Synthetic
  misc_feature (1)..(21): double stranded
  misc_feature (20)..(21): Sequence is DNA/RNA hybrid wherein N = T
cguacgcggaauacuucgann

SEQ ID NO: 11 - DNA, 31 bp, Artificial Sequence
  single stranded DNA
  misc_feature (1)..(31): Primer 1
gtctagacatatggttcagaacacctctgca

SEQ ID NO: 12 - DNA, 30 bp, Artificial Sequence
  single stranded DNA
  misc_feature (1)..(30): Primer 2
tgcctcgagcttcaattctctaattaactt

SEQ ID NO: 13 - DNA, 31 bp, Artificial Sequence
  single stranded DNA
  misc_feature (1)..(31): Primer 3
gtctagacatatggtagagaatagtcaagtg

SEQ ID NO: 14 - DNA, 30 bp, Artificial Sequence
  single stranded DNA
  misc_feature (1)..(30): Primer 4
tgcctcgagatcttcatttactttagcaat

SEQ ID NO: 15 - protein, 373 aa, Homo Sapiens
MASQPNSSAKKKEEKGKNIQVVVRCRPFNLAERKASAHSIVECDPVRKEVSVRTGGLADKSSRKTYTFDMVFGASTKQIDVYRSVVCPILDEVIMGYNCTIFAYGQTGTGKTFTMEGERSPNEEYTWEEDPLAGIIPRTLHQIFEKLTDNGTEFSVKVSLLEIYNEELFDLLNPSSDVSERLQMFDDPRNKRGVIIKGLEEITVHNKDEVYQILEKGAAKRTTAATLMNAYSSRSHSVFSVTIHMKETTIDGEELVKIGKLNLVDLAGSENIGRSGAVDKRAREAGNINQSLLTLGRVITALVERTPHVPYRESKLTRILQDSLGGRTRTSIIATISPASLNLEETLSTLEYAHRAKNILNKPEVNQHHHHHH

SEQ ID NO: 16 - protein, 365 aa, Homo Sapiens
MPINKSEKPESCDNVKVVVRCRPLNEREKSMCYKQAVSVDEMRGTITVHKTDSSNEPPKTFTFDTVFGPESKQLDVYNLTARPIIDSVLEGYNGTIFAYGQTGTGKTFTMEGVRAIPELRGIIPNSFAHIFGHIAKAEGDTRFLVRVSYLEIYNEEVRDLLGKDQTQRLEVKERPDVGVYIKDLSAYVVNNADDMDRIMTLGHKNRSVGATNMNEHSSRSHAIFTITIECSEKGIDGNMHVRMGKLHLVDLAGSERQAKTGATGQRLKEATKINLSLSTLGNVISALVDGKSTHVPYRNSKLTRLLQDSLGGNSKTMMCANIGPADYNYDETISTLRYANRAKNIKNKARVDKLAAALEHHHHHH

SEQ ID NO: 17 - protein, 352 aa, Homo Sapiens
MADLAECNIKVMCRFRPLNESEVNRGDKYIAKFQGEDTVVIASKPYAFDRVFQSSTSQEQVYNDCAKKIVKDVLEGYNGTIFAYGQTSSGKTHTMEGKLHDPEGMGIIPRIVQDIFNYIYSMDENLEFHIKVSYFEIYLDKIRDLLDVSKTNLSVHEDKNRVPYVKGCTERFVCSPDEVMDTIDEGKSNRHVAVTNMNEHSSRSHSIFLINVKQENTQTEQKLSGKLYLVDLAGSEKVSKTGAEGAVLDEAKNINKSLSALGNVISALAEGSTYVPYRDSKMTRILQDSLGGNCRTTIVICCSPSSYNESETKSTLLFGQRAKTIKNTVCVNVELTAVDKLAAALEHHHHHH

SEQ ID NO: 18 - protein, 355 aa, Homo Sapiens
MAETNNECSIKVLCRFRPLNQAEILRGDKFIPIFQGDDSVVIGGKPYVFDRVFPPNTTQEQVYHACAMQIVKDVLAGYNGTIFAYGQTSSGKTHTMEGKLHDPQLMGIIPRIARDIFNHIYSMDENLEFHIKVSYFEIYLDKIRDLLDVTKTNLSVHEDKNRVPFVKGCTERFVSSPEEILDVIDEGKSNRHVAVTNMNEHSSRSHSIFLINIKQENMETEQKLSGKLYLVDLAGSEKVSKTGAEGAVLDEAKNINKSLSALGNVISALAEGTKSYVPYRDSKMTRILQDSLGGNCRTTMFICCSPSSYNDAETKSTLMFGQRAKTIKNTASVNLELTAEVDKLAAALEHHHHHH

SEQ ID NO: 19 - protein, 355 aa, Homo Sapiens
MAEEGAVAVCVRVRPLNSREESLGETAQVYWKTDNNVIYQVDGSKSFNFDRVFHGNETTKNVYEEIAAPIIDSAIQGYNGTIFAYGQTASGKTYTMMGSEDHLGVIPRAIHDIFQKIKKFPDREFLLRVSYMEIYNETITDLLCGTQKMKPLIIREDVNRNVYVADLTEEVVYTSEMALKWITKGEKSRHYGETKMNQRSSRSHTIFRMILESREKGEPSNCEGSVKVSHLNLVDLAGSERAAQTGAAGVRLKEGCNINRSLFILGQVIKKLSDGQVGGFINYRDSKLTRILQNSLGGNAKTRIICTITPVSFDETLTALQFASTAKYMKNTPYVNEVSTVDKLAAALEHHHHHH

SEQ ID NO: 20 - protein, 448 aa, Homo Sapiens
MARAKTPRKPTVKKGSQTNLKDPVGVYCRVRPLGFPDQECCIEVINNTTVQLHTPEGYRLNRNGDYKETQYSFKQVFGTHTTQKELFDVVANPLVNDLIHGKNGLLFTYGVTGSGKTHTMTGSPGEGGLLPRCLDMIFNSIGSFQAKRYVFKSNDRNSMDIQCEVDALLERQKREAMPNPKTSSSKRQVDPEFADMITVQEFCKAEEVDEDSVYGVFVSYIEIYNNYIYDLLEEVPFDPIKPKPPQSKLLREDKNHNMYVAGCTEVEVKSTEEAFEVFWRGQKKRRIANTHLNRESSRSHSVFNIKLVQAPLDADGDNVLQEKEQITISQLSLVDLAGSERTNRTRAEGNRLREAGNINQSLMTLRTCMDVLRENQMYGTNKMVPYRDSKLTHLFKNYFDGEGKVRMIVCVNPKAEDYEENLQVMRFAEVTQEVDKLAAALEHHHHHH

SEQ ID NO: 21 - protein, 365 aa, Homo Sapiens
MSGASVKVAVRVRPFNSRETSKESKCIIQMQGNSTSIINPKNPKEAPKSFSFDYSYWSHTSPEDPCFASQNRVYNDIGKEMLLHAFEGYNVCIFAYGQTGAGKSYTMMGKQEESQAGIIPQLCEELFEKINDNCNEEMSYSVEVSYMEIYCERVRDLLNPKNKGNLRVREHPLLGPYVEDLSKLAVTSYTDIADLMDAGNKARTVAATNMNETSSRSHAVFTIVFTQKKHDNETNLSTEKVSKISLVDLAGSERADSTGAKGTRLKEGANINKSLTTLGKVISALAEVDNCTSKSKKKKKTDFIPYRDSVLTWLLRENLGGNSRTAMVAALSPADINYDETLSTLRYADRVDKLAAALEHHHHHH

SEQ ID NO: 22 - protein, 464 aa, Homo Sapiens
NPVNSVRRKSCLVKEVEKMKNKREEKKAQNSEMRMKRAQEYDSSFPNWEFARMIKEFRATLECHPLTMTDPIEEHRICVCVRKRPLNKQELAKKEIDVISIPSKCLLLVHEPKLKVDLTKYLENQAFCFDFAFDETASNEVVYRFTARPLVQTIFEGGKATCFAYGQTGSGKTHTMGGDLSGKAQNASKGIYAMASRDVFLLKNQPCYRKLGLEVYVTFFEIYNGKLFDLLNKKAKLRVLEDGKQQVQVVGLQEHLVNSADDVIKMIDMGSACRTSGQTFANSNSSRSHACFQIILRAKGRMHGKFSLVDLAGNERGADTSSADRQTRMEGAEINKSLLALKECIRALGQNKAHTPFRESKLTQVLRDSFIGENSRTCMIATISPGISSCEYTLNTLRYADRVKELSPHSGPSGEQLIQMETEEMEACSNGALIPGNLSKEEEELSSQMSSFNEAMTQIRELEE

SEQ ID NO: 23 - DNA, 21 bp, Artificial Sequence
  Synthetic
  misc_feature (1)..(21): double stranded
  misc_feature (20)..(21): Sequence is DNA/RNA hybrid wherein N = T
guuggcuagaauugggaaann

SEQ ID NO: 24 - DNA, 21 bp, Artificial Sequence
  Synthetic
  misc_feature (1)..(21): double stranded
  misc_feature (20)..(21): Sequence is DNA/RNA hybrid wherein N = T
ggacaacugcagcuacucunn

(19) United States
(12) Patent Application Publication
(10) Pub. No.: US 2007/0134660 A1
(43) Pub. Date: Jun. 14, 2007

(54) Methods for identifying modulators of kinesin activity

(75) Inventors: Mao Mao, Richmond, WA (US); Peter S. Linsley, Seattle, WA (US); Carolyn Buser, Harleysville, PA (US); Christopher Gary Marshall, Framingham, MA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignees: ROSETTA INPHARMATICS LLC, Seattle, WA (US); MERCK & CO., INC., Rahway, NJ (US)

(21) Appl. No.: 10/558,463

(22) PCT Filed: May 28, 2004
(86) PCT No.: PCT/US04/17234

§ 371 Date: Feb. 7, 2007

Related U.S. Application Data
(60) Provisional application No. 60/474,488, filed on May 30, 2003.
(60) Provisional application No. 60/475,873, filed on Jun. 3, 2003.
(60) Provisional application No. 60/553,838, filed on Mar. 17, 2004.

(51) Int. Cl. C12Q 1/68; G01N 33/574; A61K 31/175

(52) U.S. Cl. 435/6; 435/7.23; 514/582; 514/590

(57) ABSTRACT

In a first aspect, the invention provides methods for screening for modulators of a target protein, comprising the steps of contacting a target protein with a candidate agent and determining whether the candidate agent modulates the activity of the target protein, wherein the target protein comprises a sequence that has more than 80% amino acid sequence identity to KIF14 (SEQ ID NO:2) or the KIF14 motor domain (SEQ ID NO:3). In a second aspect, the invention provides methods for modulating cell proliferation comprising administering to a cell an effective amount of a modulator of the activity of a target protein. Some embodiments of this aspect provide methods for treating a subject with a cellular hyperproliferation disorder, such as cancer. In a third aspect, the invention provides methods for identifying candidate subjects for treatment with an inhibitor of the activity of a target protein.

### FIELD OF THE INVENTION

The invention relates to methods for identifying modulators of the activity of KIF14 and related proteins, and methods of treating conditions such as cancer using these modulators.

### BACKGROUND OF THE INVENTION

Breast cancer is the most common cancer in women and the second most common cause of cancer death in the United States. KIF14 was identified as a gene whose expression was positively correlated with a poor prognostic outcome of breast cancer, as assessed by the time interval to distant metastases in patients without tumor cells in local lymph nodes at diagnosis (van't Veer et al. (2002) Nature 415:530-536). KIF14 is a member of the kinesin family (KIF) of proteins. Kinesins are microtubule-dependent molecular motors that use the energy from ATP hydrolysis to move cargo along microtubules. Many kinesins have been shown to play important roles in cell division.

There is a need for methods to identify compounds that will be useful for inhibiting cellular proliferation and treating patients with cellular proliferation disorders, such as breast cancer. In particular, there is a need for methods for identifying modulators of the activity of a target protein such as KIF14, whose expression is associated with poor prognosis in cancer patients. The present invention addresses these needs.

### SUMMARY OF THE INVENTION

The sequence of the KIF14 cDNA is provided in SEQ ID NO:1. In a first aspect, the invention provides methods for screening for modulators of a target protein, wherein the target protein comprises a sequence that has more than 80% amino acid sequence identity to KIF14 (SEQ ID NO:2) or the KIF14 motor domain (SEQ ID NO:3). The methods comprise the steps of contacting a target protein with a candidate agent and determining whether the candidate agent modulates the activity of the target protein. Some embodiments provide methods in which (a) the target protein is contacted with the candidate agent at a first concentration and a first level of activity of the target protein is measured; and (b) the target protein is contacted with the candidate agent at a second concentration and a second level of activity of the target protein is measured, wherein a difference between the first level of activity and the second level of activity of the target protein indicates that the candidate agent modulates the activity of the target protein. In some embodiments, the target protein comprises the amino acid sequence of KIF14 (SEQ ID NO:2). The target protein may also comprise amino acids 356 to 709 encoding the KIF14 motor domain (SEQ ID NO:3) or any fragment of SEQ ID NO:3 having ATPase activity. For example, the target protein may be a protein comprising the sequence between amino acid 342 to amino acid 720 of the KIF14 protein (SEQ ID NO:4), a protein comprising the sequence between amino acid 342 to amino acid 710 of the KIF14 protein (SEQ ID NO:5), a protein comprising the sequence between amino acid 354 to amino acid 720 of the KIF14 protein (SEQ ID NO:6), or a protein comprising the sequence between amino acid 354 to amino acid 710 of the KIF14 protein (SEQ ID NO:7).

The target protein may be contacted with the candidate agent in vivo or in vitro. For example, the methods may also comprise expressing the target protein in a cell. The assays used for measuring the activity of the target protein include, but are not limited to, ATPase assays, binding assays, microtubule-binding assays, and microtubule-gliding assays, cell proliferation assays, cell viability assays, cell cycle distribution assays, and cell death assays. These assays may use fluorescence, luminescence, radioactivity, or absorbance for determining whether the candidate agent modulates the activity of the target protein. In some embodiments, a high throughput screening assay is used for determining whether the candidate agent modulates the activity of the target protein.

In a second aspect, the invention provides methods of modulating cell proliferation, comprising administering to a cell an effective amount of a modulator of the activity of a target protein, wherein the target protein comprises a sequence that has more than 80% sequence identity to the sequence provided in SEQ ID NO:2 or SEQ ID NO:3. The modulators may be administered to cells in vitro, such as in tissue culture, or in vivo, such as to a subject. In some embodiments, the target protein comprises the amino acid sequence provided in SEQ ID NO:2 or SEQ ID NO:3, or any fragment thereof having ATPase activity. The modulator of the activity of the target protein may be an inhibitor, such as an inhibitor of target protein expression or an inhibitor of microtubule-dependent ATP hydrolysis by the target protein. In some embodiments, the modulator is an RNA inhibitor, for example, a KIF14 RNA inhibitor comprising the sequence provided in SEQ ID NO:8, SEQ ID NO:9, or SEQ ID NO:23. In some embodiments, the modulator is an inhibitor of microtubule-dependent ATP hydrolysis by the target protein. Inhibitors of microtubule-dependent ATP hydrolysis by the target protein include, but are not limited to, small organic compounds, such as semicarbazones and thiosemicarbazones. For example, the inhibitor may be an aryl thiosemicarbazone.

Some embodiments of this aspect of the invention provide methods for treating a subject with a cellular hyperproliferation disorder, such as cancer. These methods comprise administering to a subject with a cellular hyperproliferation disorder, such as breast cancer, a therapeutically effective amount of an inhibitor of the activity of a target protein, wherein the target protein comprises a sequence that has more than 80% sequence identity to the sequence provided in SEQ ID NO:2 or SEQ ID NO:3. Some embodiments of this aspect of the invention provide methods of treating a subject with a cellular hyperproliferation disorder by administering therapeutically effective amounts of a known therapeutic agent and an inhibitor of the activity of a target protein to the subject.

In a third aspect, the invention provides methods for identifying candidate subjects for treatment with a modulator of the activity of a target protein, wherein the target protein comprises a sequence that has more than 80% sequence identity to the sequence provided in SEQ ID NO:2 or SEQ ID NO:3. These methods comprise the steps of: (a) measuring the level of expression of a target protein in sample cells of a subject and (b) identifying the subject as a candidate subject for treatment with a modulator of the activity of a target protein if the level of expression of the target protein in the sample cells is significantly different than in control cells. In some embodiments, the target protein comprises the amino acid sequence provided in SEQ ID NO:2 or SEQ ID NO:3, or any fragment thereof having ATPase activity. The level of expression of the target protein in sample cells may be determined at the level of mRNA or at the level of protein. The methods may further comprise the step of treating the candidate subject with a modulator of the activity of the target protein.

Some embodiments provide methods for identifying candidate subjects for treatment with an inhibitor of the activity of the a target protein by (a) measuring the level of expression of a target protein in abnormally proliferating cells of a subject and (b) identifying the subject as a candidate subject for treatment with an inhibitor of the activity of a target protein if the level of expression of the target protein in the abnormally proliferating cells is significantly higher than in control cells. The methods may further comprise the step of treating the candidate subject with an inhibitor of the activity of the target protein.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows the patterns of gene regulation in various cell lines treated with a panel of growth factors for increasing amounts of time, as described in EXAMPLE 2. Tumor (MCF7, HT29) and normal (HMEC, SKMC) cells were serum-starved and then stimulated with growth factors heregulin, insulin, IGF1, FGF and EGF for 0.5, 2, 6, 18 or 24 hrs. White bars indicate up-regulated genes; black bars indicate down regulated genes. Each row represents cells treated with a different growth factor, with time of treatment increasing in the upward direction. Data were clustered with kinesin sequences present on the hu25k array. The kinesins annotated in LocusLink as having mitotic function are indicated with diamonds; those annotated as transport functions with squares; and one kinesin annotated with both mitotic and transport functions with a circle. The arrow indicates KIF14.

### DETAILED DESCRIPTION OF THE PREFERRED EMBODIMENT

The present invention provides methods for screening for modulators of a target protein. The invention also provides methods for inhibiting cell proliferation and methods of treating a subject with a cellular proliferation disorder by administering an effective amount of an inhibitor of a target protein. Furthermore, the invention provides methods for identifying candidate subjects for treatment with inhibitors of a target protein. In some embodiments of the methods, the target protein is the KIF14 protein (SEQ ID NO:2). In some embodiments, the target protein is a protein comprising a sequence that has more than 80% amino acid sequence similarity to the KIF14 protein (SEQ ID NO:2) or to the motor domain of KIF14 (SEQ ID NO:3).

The expression of the KIF14 transcript (SEQ ID NO:1) was found to be positively correlated with a poor prognostic outcome of breast cancer, as assessed by the time interval to distant metastases in patients without tumor cells in local lymph nodes at diagnosis (van't Veer et al. (2002) Nature 415:530-536). The KIF14 gene encodes a protein (SEQ ID NO:2) with a putative kinesin motor domain (MD) (SEQ ID NO:3). As used herein, the term “motor domain” refers to the domain of a target protein that confers membership in the kinesin superfamily of motor proteins (see, e.g., Vale & Fletterick (1997) Annu. Rev. Cell Dev. Biol. 13:745-77). The expression of the KIF14 transcript (SEQ ID NO:1) is elevated in tumor cells, as described in EXAMPLE 1. The pattern of KIF14 expression in cell lines treated with growth factor is similar to that of mitotic kinesins, as described in EXAMPLE 2. In addition, the accumulation of KIF14 mRNA during mitosis as well as the dynamic cellular localization of KIF14 protein during mitosis is similar to that observed for mitotic kinesins, as described in EXAMPLE 3. Moreover, reducing KIF14 expression in cells results in growth inhibition and cell death, as described in EXAMPLE 4. Specifically, reduction of KIF14 expression is associated with aberrant cytokinesis, as described in EXAMPLES 5 and 6. The effect of KIF14 depletion on cytokinesis is more pronounced in tumor cells than in normal cells, as shown in EXAMPLE 6.

In a first aspect, the invention provides methods for screening for modulators of a target protein, wherein the target protein comprises a sequence that has more than 80% amino acid sequence identity to KIF14 (SEQ ID NO:2) or the KIF14 motor domain (SEQ ID NO:3). The methods comprise the steps of contacting a target protein with a candidate agent and determining whether the candidate agent modulates the activity of the target protein.

As used herein, the term “target protein” refers to a protein that has one or more of the biological activities of KIF14, including, but not limited to, microtubule stimulated ATPase activity, as tested, for example, in an ATPase assay. “ATPase activity” refers to the ability to hydrolyze ATP. Biological activity can also be demonstrated in a microtubule gliding assay or a microtubule binding assay. Other biological activities of target proteins may include polymerization/depolymerization (effects on microtubule dynamics), binding to other proteins of the spindle, binding to proteins involved in cell-cycle control, or serving as a substrate to other enzymes, such as kinases or proteases and specific kinesin cellular activities, such as involvement in chromosome segregation. The term “protein” refers to a compound that comprises at least two covalently linked amino acids. The target proteins may be from eukaryotes or prokaryotes, such as from mammals, fungi, bacteria, insects, plants, and viruses.

In addition, the target proteins used in the methods of the invention are proteins ith a sequence that has more than 80% amino acid sequence identity (such as more than 90% sequence identity, more than 95% amino acid sequence identity, or more than 99% sequence identity) to KIF14 (SEQ ID NO:2) or the KIF1414 motor domain (SEQ ID NO:3). The terms “identical” or percent “identity”, in the context of two or more amino acid sequences, refer to two or more sequences or subsequences that are the same or have a specified percentage of amino acid residues that are the same, when compared and aligned for maximum correspondence over a comparison window, as measured using one of the following sequence comparison algorithms or by manual alignment and visual inspection.

It is recognized that amino acid positions that are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. The scoring of conservative substitutions can be calculated according to, for example, the algorithm of Meyers & Millers (1988) Computer Applic. Biol. Sci. 4:11-17.

A “comparison window” includes reference to a segment of contiguous positions, such as between about 25 and about 600 positions, or between about 50 to 200 positions, or between about 100 and 150 positions, over which a sequence may be compared to a reference sequence of the same number of contiguous positions after the two sequences are optimally aligned. Methods of alignment of sequences for comparison are well-known in the art. Optimal alignment of sequences for comparison can be conducted, for example, by a local homology algorithm (Smith & Waterman (1981) Adv. Appl. Math. 2:482), by a global alignment algorithm (Needleman & Wunsch (1970) J. Mol. Biol. 48:443), by search for similarity methods (Pearson & Lipman (1988) Proc. Natl. Acad. Sci. U.S.A. 85:2444; Altschul et al. (1997) Nucl. Acids Res. 25(17):3389-402), by computerized implementations of these algorithms (e.g., GAP, BESTFIT, FASTA, and BLAST in the Wisconsin Genetics Software Package, Genetics Computer Group, 575 Science Dr., Madison, Wis.), typically using the default settings, or by manual alignment and visual inspection (see, e.g., Current Protocols in Molecular Biology (1994) Ausubel et al., eds.). For example, BLAST protein searches can be performed using the XBLAST program, score=50, wordlength=3 to obtain amino acid sequences that are more than 80% identical to the amino acid sequence of KIF14 (SEQ ID NO:2) or the KIF14 motor domain (SEQ ID NO:3).

One example of a useful algorithm implementation is PILEUP. PILEUP creates a multiple sequence alignment from a group of related sequences using progressive, pairwise alignments. It can also plot a dendrogram showing the clustering relationships used to create the alignment. PILEUP uses a simplification of the progressive alignment method of Feng & Doolittle (1987) J. Mol. Evol. 35:351-60. The method used is similar to the method described by Higgins & Sharp (1989) CABIOS 5:151-3. The multiple alignment procedure begins with the pairwise alignment of the two most similar sequences, producing a cluster of two aligned sequences. This cluster can then be aligned to the next most related sequence or cluster of aligned sequences. Two clusters of sequences can be aligned by a simple extension of the pairwise alignment of two individual sequences. A series of such pairwise alignments that includes increasingly dissimilar sequences and clusters of sequences at each iteration produces the final alignment.

The definition of target proteins also include proteins encoded by nucleic acid sequences that hybridize to the sequence encoding KIF14 (SEQ ID NO:1) to form a heteroduplex with a Tₘ that is within 20° C. of that of KIF14 (SEQ ID NO:1) homoduplex. The melting temperature of a DNA duplex is calculated using the formula:
Tₘ=81.5+16.6(log₁₀[Na⁺]+0.41(fraction G+C)−0.63(% formamide)−(600/l)
where l is the length of the hybrid in basepairs (Sambrook et al. (1989) Molecular Cloning: A Laboratory Manual, 2d ed., Cold Spring Harbor Laboratory, Cold Spring Harbor, N.Y., page 9.51). This equation applies to the “reversible” Tₘ that is defined by optical measurement of the hyperchromicity at OD₂₅₇. The melting temperature decreases by 1-1.5° C. for every 1% decrease in sequence identity (Sambrook et al. (1989) Molecular Cloning: A Laboratory Manual, 2d ed., Cold Spring Harbor Laboratory, Cold Spring Harbor, N.Y., page 9.51).

Also included within the definition of target proteins of the present invention are amino acid sequence variants of wild-type target proteins. These variants fall into one or more of three classes: substitutional, insertional or deletional variants. These variants may be prepared by site-specific mutagenesis of nucleotides in the DNA encoding the target protein. Site-specific mutagenesis may be performed using cassette or PCR mutagenesis or other techniques well known in the art, to produce DNA encoding the variant, and thereafter expressing the DNA in recombinant cell culture. Variant target protein fragments having up to about 100-150 amino acid residues may be prepared by invitro synthesis using established techniques. Amino acid sequence variants are characterized by the predetermined nature of the variation, a feature that sets them apart from naturally occurring allelic or interspecies variation of the target protein amino acid sequence. The variants typically exhibit the same qualitative biological activity as the naturally occurring analogue, although variants can also be selected which have modified properties. Conservative substitution tables providing functionally similar amino acids are well known in the art (Henikoff & Henikoff (1992) Proc. Natl. Acad. Sci. U.S.A. 89:10915-9).

Amino acid substitutions are typically of single residues. Insertions usually will be on the order of from about 1 to about 20 amino acids, although considerably longer insertions may be tolerated. Deletions range from about 1 to about 20 residues, although in some cases, deletions may be much longer. Substitutions, deletions, and insertions or any combinations thereof may be used to arrive at a final derivative.

Accordingly, in some embodiments of the methods, the target protein comprises the KIF14 protein (SEQ ID NO:2). In other embodiments, the target protein comprises a portion of the KIF14 protein (SEQ ID NO:2) encoding the KIF14 motor domain (SEQ ID NO:3), or fragments thereof that have microtubule-dependent ATPase activity. For example, the target protein may be a protein comprising the sequence between amino acid 342 to amino acid 720 of the KIF14 protein (SEQ ID NO:4). Alternatively, the target protein may be a protein comprising the sequence between amino acid 342 to amino acid 710 of the KIF14 protein (SEQ ID NO:5), a protein comprising the sequence between amino acid 354 to amino acid 720 of the KIF14 protein (SEQ ID NO:6), or a protein comprising the sequence between amino acid 354 to amino acid 710 of the KIF14 protein (SEQ ID NO:7).

The target proteins used in the methods of the invention are typically expressed using an expression system and purified. An expression system includes expression vectors and host cells. The expression vectors may be either self-replicating extrachromosomal vectors or vectors which integrate into a host genome. Generally, expression vectors include transcriptional and translational regulatory nucleic acid operably linked to the nucleic acid encoding the target protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers. Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Operably linked DNA sequences may be contiguous or non-contiguous. Linking may be accomplished by ligation, for example by ligation at convenient restriction sites. If such sites do not exist, blunt-end ligation and/or synthetic oligonucleotide adaptors or linkers may be used. The transcriptional and translational regulatory nucleic acid will generally be appropriate to the host cell used to express the target protein; for example, transcriptional and translational regulatory nucleic acid sequences from Bacillus are preferably used to express the target protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. Promoter sequences encode either constitutive or inducible promoters. The promoters may be either naturally occurring promoters or hybrid promoters. Hybrid promoters, which combine elements of more than one promoter, are also known in the art.

An expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to a sequence in the host cell genome, and preferably two homologous sequences that flank the expression construct. The integrating vector may be directed to a specific locus in the host cell by selecting the appropriate homologous sequence for inclusion in the vector. Constructs for integrating vectors are well known in the art.

In addition, an expression vector typically contains a selectable marker gene to allow the selection of transformed host cells. Selection genes are well known in the art and will vary with the host cell used.

The target proteins used in the present invention may be produced by culturing a host cell transformed with an expression vector containing nucleic acid encoding a target protein, under the appropriate conditions to induce or cause expression of the target protein. The conditions appropriate for target protein expression will vary with the choice of the expression vector and the host cell, and will be easily ascertained by one skilled in the art using routine experimentation. For example, the growth and proliferation of the host cell may be optimized for the use of constitutive promoters in the expression vector, and appropriate growth conditions for induction are provided for use of an inducible promoter. In addition, in some embodiments, the timing of the harvest is important, for example, when using baculoviral systems.

Appropriate host cells include yeast, bacteria, archaebacteria, fungi, and insect and animal cells, including mammalian cells. Of particular interest are Drosophila melanogaster cells, Saccharomyces cerevisiae and other yeasts, E. coli, Bacillus subtilis, Sf9 cells, C129 cells, 293 cells, Neurospora, BHK, CHO, COS, HeLa cells, THP1 cell line (a macrophage cell line), and human cells and cell lines.

Accordingly, in some embodiments, the target proteins are expressed in mammalian cells. Mammalian expression systems are also known in the art, and include retroviral systems. Promoters from viral genes are frequently used in mammalian expression systems, because the viral genes are often highly expressed and have a broad host range. Examples include the SV40 early promoter, mouse mammary tumor virus LTR promoter, adenovirus major late promoter, herpes simplex virus promoter, and the CMV promoter. Typically, transcription termination and polyadenylation sequences recognized by mammalian cells are regulatory regions located 3′ to the translation stop codon and thus, together with the promoter elements, flank the coding sequence. Examples of transcription terminator and polyadenylation signals include those derived from SV40.

The methods of introducing exogenous nucleic acid into mammalian hosts, as well as other hosts, are well known in the art, and will vary with the host cell used. Techniques include dextran-mediated transfection, calcium phosphate precipitation, polybrene mediated transfection, protoplast fuision, electroporation, viral infection, encapsulation of the polynucleotide(s) in liposomes, and direct microinjection of the DNA into nuclei.

In some embodiments, the target proteins are expressed in bacterial systems. Bacterial expression systems are well known in the art. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the target protein in bacteria. The target protein is either secreted into the growth media (gram-positive bacteria) or into the periplasmic space, located between the inner and outer membrane of the cell (gram-negative bacteria). The expression vector may also include an epitope tag providing for affinity purification of the target protein. The bacterial expression vector may also include a selectable marker gene to allow for the selection of bacterial strains that have been transformed. Suitable selection genes include genes that render the bacteria resistant to drugs such as ampicillin, chloramphenicol, erythromycin, kanamycin, neomycin and tetracycline. Selectable markers also include biosynthetic genes, such as those in the histidine, tryptophan, and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others. The bacterial expression vectors are transformed into bacterial host cells using techniques well known in the art, such as calcium chloride treatment, electroporation, and others. An exemplary method for expressing KIF14 motor domain proteins using a bacterial expression system is described in EXAMPLE 7.

Target proteins may also be produced in insect cells. Expression vectors for the transformation of insect cells, and in particular, baculovirus-based expression vectors, are well known in the art. In addition, target proteins may be produced in yeast cells. Yeast expression systems are well known in the art, and include expression vectors for Saccharomyces cerevisiae, Candida albicans and C. maltosa, Hansenula polymopha, Kluyveromyces fragilis and K. lactis, Pichia guillerimondii and P. pastoris, Schizosaccharomyces pombe, and Yarrowia lipolytica.

The target protein may also be made as a fusion protein, using techniques well known in the art. For example, the target protein may be made as a fusion protein to increase expression or to link it with a tag polypeptide that provides an epitope to which an anti-tag antibody can selectively bind. Exemplary tags include the myc epitope and 6-histidine. The epitope tag is generally placed at the amino- or carboxyl-terminus of the target protein. The presence of such epitope-tagged forms of a target protein can be detected using an antibody against the tag polypeptide. Thus, the epitope tag enables the target proteins to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 (Field et al. (1988) Mol. Cell. Biol. 8:2159-65); the c-myc tag and the 8F9, 3C7, 6E10, G4, B7 and 9E10 antibodies thereto (Evan et al. (1985) Mol. Cell. Biol. 5:3610-6); and the Herpes Simplex virus glycoprotein D (gD) tag and its antibody (Paborsky et al. (1990) Prot. Eng. 3(6):547-53). Other tag polypeptides include the Flag-peptide (Hopp et al. (1988) BioTechnol. 6:1204-10); the KT3 epitope peptide (Martin et al. (1992) Science 255:192-4); tubulin epitope peptide (Skinner et al. (1991) J. Biol. Chem. 266:15163-6); and the T7 gene 10 protein peptide tag (Lutz-Freyermuth et al. (1990) Proc. Natl. Acad. Sci. U.S.A. 87:6393-7).

The target proteins used in the methods of the invention may be labeled. As used herein, the term “labeled” refers to the attachment of at least one element, isotope or chemical compound to enable the detection of the target protein. A label is any composition detectable by spectroscopic, photochemical, biochemical, immunochemical, electrical, optical, or chemical means. Thus, labels may be isotopic labels; which may be radioactive or heavy isotopes, immune labels, which may be antibodies or antigens; and colored or fluorescent dyes. The labels may be incorporated into the target proteins at any position. For example, the label should be capable of producing, either directly or indirectly, a detectable signal. The detectable moiety may be a radioisotope, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for attaching the label to the target protein may be employed.

Covalent modifications of target proteins are included within the scope of this invention. One type of covalent modification includes reacting targeted amino acid residues of a target protein with an organic derivatizing agent that is capable of reacting with selected side chains or the N- or C-terminal residues of a target protein. Derivatization with bifunctional agents is useful, for instance, for crosslinking a target protein to a *vater-insoluble support matrix or surface for use in screening assays. Commonly used crosslinking agents include, but are not limited to, 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

The target protein may be purified or isolated after expression. The terms “isolated” “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. The term “purified” denotes that a protein gives rise to essentially one band in an electrophoretic gel. For example, it means that the protein is at least 85% pure, such as at least 95% pure, such as at least 99% pure.

Target proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the target protein may be purified using a standard anti-KIF14 antibody column (see, e.g., KIF14 antibody ab3746, Abcam). Ultrafiltration and diafiltration techniques, in conjunction with protein concentration, are also useful. Suitable purification techniques are standard in the art (see, e.g., Scopes (1982) Protein Purification, Springer-Verlag, NY). The degree of urification necessary will vary depending on the use of the target protein. In some instances no purification may be necessary. Exemplary protocols for purifying target proteins for use in the methods of the invention are provided in EXAMPLES 7 and 8.

In the first step of the methods of this aspect of the invention, the target protein is contacted with a candidate agent. Candidate agents may encompass numerous chemical classes. Typically they are organic molecules, preferably small organic compounds having a molecular weight of more than 100 and less than about 2500 daltons. Small molecules are further defmed herein as having a molecular weight of between 150 daltons and 2000 daltons, such as less than 1500, or less than 1200, or less than 1000, or less than 750, or less than 500 daltons. Thus, a small molecule may have a molecular weight of about 100 to 200 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs, or combinations thereof.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, and amidification, to produce structural analogs.

The second step of the methods comprises determining whether the candidate agent modulates the activity of the target protein. As used herein, the term “modulates the activity of the target protein” refers to any change in the activity of the target protein, such as a decrease or an increase in the activity. Typically, samples or assays are treated with a candidate agent at a test and control concentration. The control concentration may be zero. If there is a change in target protein activity between the two concentrations, this change indicates that the candidate agent modulates the activity of the target protein. Thus, some embodiments provide methods in which (a) the target protein is contacted with the candidate agent at a first concentration and a first level of activity of the target protein is measured; and (b) the target protein is contacted with the candidate agent at a second concentration and a second level of activity of the target protein is measured, wherein a difference between the first level of activity and the second level of activity of the target protein indicates that the candidate agent modulates the activity of the target protein. A difference in activity, which can be an increase or decrease, may be a change of at least 20% to 50%, such as at least 50% to 75%, such as at least 75% to 100%, such as at least 150% to 200%, such as at least 200% to 1000%, compared to a control. Additionally, a difference in activity can be indicated by a change in binding specificity or substrate.

The activity of the target protein may be measured using in vitro assays and purified or partially purified proteins. The activity of the target protein may also be measured using in vivo assays by expressing the target protein in cells. The assays used may be multi-time-point (kinetic) assays, with at least two data points. In the case of multiple measurements, the absolute rate of the protein activity may be determined. As will be appreciated by those in the art, the components in the assay may be added in buffers and reagents to assay target protein activity and give optimal signals. Moreover, to allow kinetic measurements the incubation periods are typically optimized to give adequate detection signals over the background.

Assays for measuring the activity of the target protein include measuring ATPase activity, microtubule-gliding, microtubule-polymerization/depolymerizing activity (effects on microtubule dynamics), and binding activities, such as microtubule-binding, binding to proteins of the spindle, binding to proteins involved in cell cycle control, or binding of nucleotide analogs (see, e.g., Kodama et al. (1986) J. Biochem. 99:1465-72; Stewart et al. (1993) Proc. Natl. Acad. Sci. U.S.A. 90:5209-13; Lombillo et al. (1995) J. Cell Biol. 128:107-15; Vale et al. (1985) Cell 42:39-50). In the case that the target protein used has another specific activity, such as involvement in mitosis or axonal transport, specific assays for those activities can be used. Exemplary assays are described below.

In some embodiments, the assay used to measure the activity of the target protein comprises measuring ATPase activity, as described in EXAMPLES 7-9. Thus, ADP or phosphate is used as a readout for target protein activity. In these embodiments, the target protein is contacted with the candidate agents under conditions that allow production of ADP or phosphate by the target protein and the effect of the candidate agents on the production of ADP or phosphate by the target protein is measured. Conditions that allow production of ADP or phosphate by the target protein are conditions under which the reaction which produces ADP or phosphate would normally occur in the absence of a candidate agent that modulates the activity of the target protein.

The production of ADP or phosphate may be measured enzymatically. There are a number of enzymatic reactions known in the art which use ADP as a substrate. For example, kinase reactions, such as pyruvate kinase reactions are well known and allow the regeneration of ATP (see, e.g., Greengard (1956) Nature 178:632-4). The level of activity of the enzymatic reaction may be determined directly. For example, in a pyruvate kinase reaction, pyruvate or ATP can be measured by conventional methods known in the art. The level of activity of the enzymatic reaction which uses ADP as a substrate may also be measured indirectly by being coupled to another reaction, such as a lactate dehydrogenase reaction. Measurement of enzymatic reactions by coupling is known in the art (see, e.g., Greengard (1956) Nature 178:632-4).

Furthermore, there are a number of reactions which utilize phosphate, for example a purine nucleoside phosphorylase reaction. This reaction may be measured directly by conventional methods known in the art. The reaction may also be measured indirectly by coupling it to another reaction, such as a purine analog cleavage reaction under conditions which normally allow the cleavage of the purine analog (see, e.g., Webb (1992) Proc. Natl. Acad. Sci. U.S.A. 89:4884-7; Rieger et al. (1997) Anal. Biochem. 246:86-95; Banik et al. (1990) Biochem. J. 266:611-4. Alternatively, xanthine oxidase may be used in conjunction with purine nucleoside phosphorylase to couple phosphate production to a change in the absorbance of a substrate for xanthine oxidase (Ungerer et al. (1993) Clin. Chim. Acta. 223:149-57).

The production of ADP or phosphate may be detected non-enzymatically, for example by binding or reacting the ADP or phosphate with a detectable compound. For example, phosphomolybdate based assays, which involve conversion of free phosphate to a phosphomolybdate complex, may be used (Fiske et al. (1925) J. Biol. Chem. 66:375-400). One method of quantifying the phosphomolybdate is with malachite green. Alternatively, a fluorescently labeled form of a phosphate-binding protein, such as the E. coli phosphate-binding protein, can be used to measure phosphate by a shift in its fluorescence.

In a preferred embodiment, detection of the assay is done using a detectable label, such as an isotopic label (radioactive or heavy isotopes), magnetic, electrical, thermal; colored or luminescent dyes, enzymes, and particles such as magnetic particles. The dyes may be chromophores, phosphors, or fluorescent dyes. Typically, fluorescent signals provide a good signal-to-noise ratio for detection. Suitable dyes for use in the invention include, but are not limited to, fluorescent lanthanide complexes, including those of Europium and Terbium, fluorescein, rhodamine, tetramethylrhodamine, eosin, erythrosin, coumarin, methyl-coumarins, pyrene, Malachite green, stilbene, Lucifer Yellow, Cascade Blue, Texas Red, and derivatives thereof, and other (see also Richard P. Haughland, Molecular Probes Handbook, 6th ed.). In some embodiments, phosphate production is measured using the dye Quinaldine Red, which absorbs light at a wavelength of 540 nm when bound to inorganic phosphate, as described in EXAMPLES 7-9.

The invention provides methods of screening candidate agents for the ability to serve as modulators of target protein activity. For example, high throughput screening (HTS) systems may be used. HTS systems may include the use of robotic systems and offer the advantage that many samples can be processed in a short period of time. HTS systems are commercially available (see, e.g., Zymark Corp., Hopkinton, Mass.; Air Technical Industries, Mentor, Ohio; Beckman Instruments, Inc., Fullerton, Calif.; Precision Systems, Inc., Natick, Mass.). HTS systems typically automate entire procedures including all sample and reagent pipetting, liquid dispensing, timed incubations, and final readings of the microplate in detector(s) appropriate for the assay. These configurable systems may be customized and provide high throughput, rapid start up, and a high degree of flexibility.

A plurality of assay mixtures may be run in parallel with different candidate agent concentrations to obtain a differential response to the various concentrations. Typically, one of these concentrations serves as a negative control, that is, a candidate agent concentration of zero or below the level of detection. However, any concentration can be used as the control for comparative purposes.

HTS methods generally involve providing a library containing a large number of candidate agents. For example, combinatorial chemical libraries may be screened in one or more assays, as described herein, to identify those library members (particular chemical species or subclasses) that display a desired characteristic activity. The compounds thus identified may serve as conventional lead compounds or can themselves be used as potential or actual therapeutic compounds.

For example, candidate agents may be assayed in highly parallel fashion by using multiwell plates and by placing the candidate agents either individually in wells or testing them in mixtures. Assay components, such as for example, target proteins, protein filaments, coupling enzymes and substrates, and ATP can then be added to the wells and the absorbance or fluorescence of each well of the plate can be measured by a plate reader. A candidate agent which modulates the function of the target protein is identified by an increase or decrease in the rate of ATP hydrolysis compared to a control assay in the absence of that candidate agent.

In some embodiments of the methods of the invention, target protein activity is identified by an ATP hydrolysis assay as described above. However, it is understood that target activity can be identified by a number of assays. Such assays include microtubule gliding, depolymerization/polymerization, and any activity which requires both binding and ATPase activity. Generally motility assays involve immobilizing one component of the system (e.g., the target protein or the microtubule) and then detecting movement, or change thereof, of the other component. Thus, for example, the target protein may be immobilized (e.g., attached to a solid substrate) and the movements of microtubules may be monitored. Typically the molecule that is to be detected is labeled (e.g., with a fluorescent label) to facilitate detection. Methods of performing motility assays are well known to those of skill in the art (see, e.g., Hall et al. (1996) Biophys. J. 71:3467-76, Turner et al. (1996) Anal. Biochem. 242(1):20-5; Gittes et al. (1996) Biophys. J. 70(1):418-29; Shirakawa et al. (1995) J. Exp. Biol. 198:1809-15; Winkelmann et al. (1995) Biophys. J. 68:2444-53; Winkelmann et al. (1995) Biophys. J. 68:72S).

Moreover, if the protein used has another specific activity, such as involvement in mitosis or axonal transport, specific assays for those activities can be utilized. For example, target protein activity may be examined by determining modulation of target protein activity in vitro using cultured cells. The cells may endogenously express the target protein, or they may be engineered to express a target protein, for example by introducing a vector comprising a nucleic acid sequence encoding the target protein, as described above. The cells are treated with a candidate agent and the effect of the candidate agent on the cells is then determined either directly or by examining relevant surrogate markers.

In some embodiments, cells containing target proteins are used in candidate agent screening assays by evaluating the effect of candidate agents on cellular proliferation. Useful cell types include normal cells and cells with abnormal proliferative rates, such as tumor cells. Methods of assessing cellular proliferation are known in the art and include growth and viability assays using cultured cells. In such assays, cell populations are monitored for growth and or viability, often over time and comparing samples incubated with various concentrations of the candidate agent or without the candidate agent. Cell number may be quantified using agents such as 3-(4,5-dimethylthiazol-2-yl)-2,5-diphenyltetrazolim bromide (MTT), 3-(4,5-dimethylthiazol-2-yl)-5-(3-carboxymethoxyphenyl)-2-(4-sulfophenyl)-2H-tetrazolium (MTS) and alamarBlue™, which are converted to colored or fluorescent compounds in the presence of metabolically active cells. Alternatively, dyes that bind to cellular protein such as sulforhodamine B (SRB) or crystal violet may be used to quantify cell number. Cells may also be directly counted using a particle counter, such as a Coulter Counter manufactured by Beckman Coulter, or counted using a microscope to observe cells on a hemocytometer. Typically, cells counted using the hemocytometer are observed in a solution of trypan blue to distinguish viable from dead cells. Other methods of quantifying cell number are known to those skilled in the art. These assays may be performed on any of the cells, including those in a state of necrosis.

Moreover, apoptosis can be determined by methods known in the art. For example, markers for apoptosis are known, and TUNEL (TdT-mediated dUTP-fluorescein nick end labeling) kits can be bought commercially (e.g., Boehringer Mannheim, Cat. No. 168795). Other markers for apoptosis include caspase activity, as described in EXAMPLE 4.

The cell proliferation assays are evaluated in the presence or absence or previous or subsequent exposure to physiological signals, for example hormones, antibodies, peptides, antigens, cytokines, growth factors, action potentials, pharmacological agents including chemotherapeutics, radiation, carcinogenics, or other cells (i.e., cell-cell contacts). In addition, the cell proliferation assays may be evaluated at different stages of the cell cycle process to assess characteristics such as mitotic spindle morphology and cell cycle distribution (see, e.g., Mayer et al. (1999) Science 286:971-4)

Exemplary methods for assessing the effect of candidate agents on the growth and viability of cells expressing KIF14 are described in EXAMPLES 4 and 9. Cells with high proliferation rates, such as cancer cells, generally express high levels of KIF14, as described in EXAMPLE 1. Conversely, reducing KIF14 expression using RNA interference results in growth inhibition and cell death, as described in EXAMPLE 4. Thus, candidate agents that modulate KIF14 activity may result in a change in cell growth or viability of KIF14-expressing cells, as described in EXAMPLE 9.

Exemplary methods for assessing the effect of candidate agents on characteristics such as mitotic spindle morphology and cell cycle distribution of cells expressing KIF14 is described in EXAMPLES 5 and 6. Reduction of KIF14 expression using RNA interference results in aberrant cytokineses and the formation of binucleate cells, as described in EXAMPLES 5 and 6. Thus, candidate agents that modulate KIF14 activity may result in a cytokinetic change in KIF14-expressing cells with no or minimal effects in cells that do not express KIF14.

In some embodiments, candidate agents that modulate the activity of a target protein may be identified by using competitive binding assays. In these assays, the competitor is a binding moiety known to bind to the target protein, such as an antibody, peptide, binding partner, or ligand.

Competitive screening assays may be done by combining the target protein and a candidate agent in a first sample. A second sample comprises that candidate agent, the target protein and a compound that is known to bind to the target protein. These assays may be performed in either the presence or absence of microtubules. The binding of the candidate agent is determined for both samples, and a change, or difference in binding between the two samples indicates the presence of an agent capable of binding to the target protein and potentially modulating its activity. That is, if the binding of the candidate agent is different in the second sample relative to the first sample, the candidate agent is capable of binding to the target protein.

The candidate agent may be labeled. Either the candidate agent, or the competitor, or both, is added first to the target protein for a time sufficient to allow binding. Incubations may be performed at any temperature which facilitates optimal activity, typically between 4° C. and 40° C. Incubation periods may also be optimized to facilitate rapid high throughput screening. Typically between 0.1 and 1 hour will be sufficient. Excess reagent is generally removed or washed away. The second component is then added, and the presence or absence of the labeled component is followed, to indicate binding.

The competitor may be added first, followed by the candidate agent. Displacement of the competitor is an indication the candidate agent is binding to the target protein and thus is capable of binding to, and potentially modulating, the activity of the target protein. In this embodiment, either component can be labeled. Thus, for example, if the competitor is labeled, the presence of label in the wash solution indicates displacement by the agent. Alternatively, if the candidate agent is labeled, the presence of the label on the support indicates displacement.

Alternatively, the candidate agent may be added first, followed by the competitor. The absence of binding by the competitor may indicate the candidate agent is bound to the target protein with a higher affinity. Thus, if the candidate agent is labeled, the presence of the label on the support, coupled with a lack of competitor binding, may indicate the candidate agent is capable of binding to the target protein.

In a second aspect, the invention provides methods for modulating cell proliferation. The methods comprise administering to a cell an effective amount of a modulator of the activity of a target protein, wherein the target protein comprises a sequence that has more than 80% sequence identity to the sequence provided in SEQ ID NO:2 or SEQ ID NO:3. The target proteins used in the methods of this aspect of the invention are as described above for the methods of the first aspect of the invention. Modulators of the activity of the target protein are agents whose administration results in a change in the activity of the target protein, as defined above. For example, a modulator of the activity of a target protein may inhibit or stimulate the activity of the target protein. Modulators that may be used in this aspect of the invention may be identified by screening candidate agents, as described above for the first aspect of the invention.

Typically, administration of modulators that inhibit the activity of the target protein have the effect of inhibiting cell growth or causing cell death, as described in EXAMPLES 4 and 9. Administration of such inhibitory modulators are useful, for example, for treating conditions in which there is hyperproliferation of cells, such as cancer, restenosis, autoimmume disease, arthritis, graft rejection, inflamniatory bowl disease, or proliferation induced after medical procedures.

Conversely, modulators that increase the activity of the target protein have the effect of stimulating cell division, as described in EXAMPLE 1. Administration of such stimulatory modulators are useful, for example, for treating conditions in which there is hypoproliferation of cells or in which enhancement of cell proliferation is desired, such as during wound healing or stem cell expansion.

Some embodiments provide methods of modulating cell proliferation by administering an inhibitor of the activity of the target protein. The inhibitor may be an RNA inhibitor. The term “RNA inhibitor” refers to an inhibitory RNA that silences expression of the target protein by RNA interference (McManus & Sharp (2002) Nat. Rev. Genet. 3:737-47; Hannon (2002) Nature 418:244-51; Paddison & Hannon (2002) Cancer Cell 2:17-23). RNA interference is conserved throughout evolution, from C. elegans to humans, and is believed to function in protecting cells from invasion by RNA viruses. When a cell is infected by a dsRNA virus, the dsRNA is recognized and targeted for cleavage by an RNaseIII-type enzyme termed Dicer. The Dicer enzyme “dices” the RNA into short duplexes of 21 nucleotides, termed short-interfering RNAs or siRNAs, composed of 19 nucleotides of perfectly paired ribonucleotides with two unpaired nucleotides on the 3′ end of each strand. These short duplexes associate with a multiprotein complex termed RISC, and direct this complex to mRNA transcripts with sequence similarity to the siRNA. As a result, nucleases present in the RISC complex cleave the mRNA transcript, thereby abolishing expression of the gene product. In the case of viral infection, this mechanism would result in destruction of viral transcripts, thus preventing viral synthesis. Since the siRNAs are double-stranded, either strand has the potential to associate with RISC and direct silencing of transcripts with sequence similarity.

Recently, it was determined that gene silencing could be induced by presenting the cell with the siRNA, mimicking the product of Dicer cleavage (Elbashir et al. (2001) Nature 411:494-8; Elbashir et al. (2001) Genes Dev. 15:188-200). Synthetic siRNA duplexes maintain the ability to associate with RISC and direct silencing of mRNA transcripts, thus providing researchers with a powerful tool for gene silencing in mammalian cells. Yet another method to introduce the dsRNA for gene silencing is shRNA, for short hairpin RNA (Paddison et al. (2002) Genes Dev. 16:948-58; Brummelkamp et al. (2002) Science 296:550-3; Sui et al. (2002) Proc. Natl. Acad. Sci. U.S.A. 99:5515-20). In this case, a desired siRNA sequence is expressed from a plasmid (or virus) as an inverted repeat with an intervening loop sequence to form a hairpin structure. The resulting RNA transcript containing the hairpin is subsequently processed by Dicer to produce siRNAs for silencing. Plasmid-based shRNAs can be expressed stably in cells, allowing long-term gene silencing in cells, or even in animals (McCaffrey et al. (2002) Nature 418:38-9; xia et al. (2002) Nat. Biotech. 20:1006-10; Lewis et al. (2002) Nat. Genetics 32:107-8; Rubinson et al. (2003) Nat. Genetics 33:401-6; Tiscomia et al. (2003) Proc. Natl. Acad Sci. U.S.A. 100:1844-8). RNA interference has been successful used therapeutically to protect mice from fulminant hepatitis (Song et al. (2003) Nat. Medicine 9:347-51).

Thus, in some embodiments of the invention, cell proliferation is inhibited by administering KIF14 siRNAs, as described in EXAMPLES 4-6. The KIF14 siRNA may comprise the sequence provided in SEQ ID NO:8, SEQ ID NO:9, or SEQ ID NO:23.

In some embodiments, cell proliferation is inhibited by administering an inhibitor of microtubule-dependent ATP hydrolysis by the target protein. Exemplary inhibitors include small molecule organic compounds, such as semicarbazones and thiosemicarbazones. For example, the inhibitor may be an aryl thiosemicarbazone, as described in EXAMPLE 9. Exemplary aryl thiosemicarbazone inhibitors include, but are not limited to, 1,1′-biphenyl-4-carbaldehyde thiosemicarbazone (compound 1), 4-isopropylbenzaldehyde thiosemicarbazone (compound 2; see, e.g., U.S. Pat. No. 3,849,575), 4-cyclohexylbenzaldehyde thiosemicarbazone (compound 3), and 4-isopropyl-3-nitrobenzaldehyde thiosemicarbazone (compound 4; see, e.g., Saripinar et al. (1996) Arzneimittel-Forschung 46(II):824-8).

The modulators may be administered to a cell in vitro, such as by administering them to cells in tissue culture. Modulators may be administered to cells in vitro using conventional protocols in the art, including transfection, lipofection, microinjection, and others described above. The modulators may also be administered to cells in vivo, by administering the modulators to a subject. The term “subject” refers to a living organism, such as a plant or an animal. Exemplary subjects are mammals, such as humans. For example, the subject may be a human cancer patient. Administration of modulators to a subject is accomplished by any effective route, for example, locally, systemically, parenterally, or orally. For example, an inhibitory modulator may be injected directly into a tumor, or into a blood vessel that supplies blood to the tumor. Methods of parenteral delivery include topical, intra-arterial, subcutaneous, intramedullary, intravenous, or intranasal administration.

The amount of the modulator actually administered in the methods of this aspect of the invention is an effective amount. The term “effective amount” refers to the amount needed to produce a substantial effect. Effective amounts of the modulators administered in the methods of this aspect of the invention will generally range up to the maximally tolerated dosage, but may vary widely. The precise amounts employed will vary depending on the compound, route of administration, physical condition of the subject, and other factors. The daily dosage may be administered as a single dosage or may be divided into multiple doses for administration.

Effective amounts of the modulator may be extrapolated from dose-response curves derived from in vitro or animal model test systems. The animal model is also typically used to determine a desirable concentration range and route of administration. Such information can then be used to determine useful doses and routes for administration in humans or other mammals. The determination of an effective dose is well within the capability of those skilled in the art. Thus, the amount actually administered will be dependent upon the individual to which treatment is to be applied, and will preferably be an optimized amount such that the desired effect is achieved without significant side-effects.

Therapeutic efficacy and possible toxicity of the modulators can be determined by standard pharmaceutical procedures, in cell cultures or experimental animals (e.g., ED₅₀, the dose therapeutically effective in 50% of the population; and LD₅₀, the dose lethal to 50% of the population). The dose ratio between therapeutic and toxic effects is the therapeutic index, and it can be expressed as the ratio ED₅₀/LD₅₀. Modulatory compounds that exhibit large therapeutic indices are particularly suitable in the practice of the methods of the invention. The data obtained from cell culture assays and animal studies may be used in formulating a range of dosage for use in humans or other mammals. The dosage of such compounds lies preferably within a range of circulating concentrations that include the ED₅₀ with little or no toxicity. The dosage typically varies within this range depending upon the dosage form employed, sensitivity of the patient, and the route of administration. Thus, optimal amounts will vary with the method of administration, and will generally be in accordance with the amounts of conventional medicaments administered in the same or a similar form.

The modulators may be formulated into a composition that additionally contains suitable pharmaceutically acceptable carriers, including excipients and other compounds that facilitate administration of the modulator to a mammalian subject. Further details on techniques for formulation and administration may be found in the latest edition of Remington's Pharmaceutical Sciences (Maack Publishing Co, Easton Pa.).

Compositions for oral administration may be formulated using pharmaceutically acceptable carriers well known in the art, in dosages suitable for oral administration. Such carriers enable the compositions containing inhibitors to be formulated as tablets, pills, dragees, capsules, liquids, gels, syrups, slurries, suspensions, etc., suitable for ingestion by a subject. Compositions for oral use may be formulated, for example, in combination with a solid excipient, optionally grinding the resulting mixture, and processing the mixture of granules, after adding suitable additional compounds, if desired, to obtain tablets or dragee cores. Suitable excipients include carbohydrate or protein fillers. These include, but are not limited to, sugars, including lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins, such as gelatin and collagen. If desired, disintegrating or solubilising agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage).

Modulators for oral administration may be formulated, for example, as push-fit capsules made of gelatin, as well as soft, sealed capsules made of gelatin and a coating such as glycerol or sorbitol. Push-fit capsules may contain modulators mixed with filler or binders such as lactose or starches, lubricants such as talc or magnesium stearate, and, optionally, stabilizers. In soft capsules, modulators may be dissolved or suspended in suitable liquids, such as fatty oils, liquid paraffin, or liquid polyethylene glycol with or without stabilizers.

Compositions for parenteral administration include aqueous solutions of one or more modulators. For injection, the modulators may be formulated in aqueous solutions, such as in physiologically compatible buffers such as Hank's solution, Ringer's solution, or physiologically buffered saline. Aqueous injection suspensions may contain substances, which increase the viscosity of the suspension, such as sodium carboxymethyl cellulose, sorbitol, or dextran. Additionally, suspensions of the modulators may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents, which increase the solubility of the modulators to allow for the preparation of highly concentrated solutions.

For topical or nasal administration, penetrants appropriate to the particular barrier to be permeated are typically used in the formulation. Examples of these are 2-pyrrolidone, N-methyl-2-pyrrolidone, dimethylacetamide, dimethyl-formamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface-active agents. Keratolytic agents such as those known in the art may also be included. Examples are salicylic acid and sulfur.

The amounts of each of these various types of additives will be readily apparent to those skilled in the art, optimal amounts being the same as in other, known formulations designed for the same type of administration. Stratum corneum penetration enhancers, for example, will typically be included at levels within the range of about 0.1% to about 15%.

Compositions containing the modulators may be manufactured in a manner similar to that known in the art (e.g., by means of conventional mixing, dissolving, granulating, dragee-making, levigating, emulsifying, encapsulating, entrapping or lyophilizing processes). The compositions may also be modified to provide appropriate release characteristics, e.g., sustained release or targeted release, by conventional means (e.g., coating).

Compositions containing the modulators may be provided as a salt and can be formed with many acids, including but not limited to hydrochloric, sulfuric, acetic, lactic, tartaric, malic, succinic, etc. Salts tend to be more soluble in aqueous or other protonic solvents than are the corresponding free base forms.

After compositions formulated to contain modulators and an acceptable carrier have been prepared, they can be placed in an appropriate container and labeled for use.

Some embodiments of this aspect of the invention provide methods of treating a subject with a cellular hyperproliferation disorder by administering a therapeutically effective amount of an inhibitor of the activity of a target protein to the subject, wherein the target protein comprising a sequence that has more than 80% sequence identity to the sequence provided in SEQ ID NO:2 or SEQ ID NO:3.

The term “cellular hyperproliferation disorders” refers to any condition in which there is excessive cellular proliferation, such as cancer, restenosis, autoimmume disease, arthritis, graft rejection, inflammatory bowl disease, or proliferation induced after medical procedures. In some embodiments, the cellular hyperproliferation disorder is cancer, including, but not limited to brain cancer, head and neck cancer, esophageal cancer, breast cancer, lung cancer, stomach cancer, pancreatic cancer, liver cancer, colorectal cancer, bladder cancer, renal cancer, prostate cancer, ovarian cancer, cervical cancer, uterine cancer, melanoma, multiple melanoma, leukemia, and lymphoma. The inhibitor administered in this embodiment of the methods may be an inhibitory RNA, such as a KIF14 siRNA. The KIF14 siRNA may comprise the sequence provided in SEQ ID NO:8, SEQ ID NO:9, or SEQ ID NO:23. The inhibitor administered may also be an inhibitor of microtubule-dependent ATP hydrolysis by the target protein. Exemplary inhibitors include small molecule organic compounds, such as semicarbazones and thiosemicarbazones. For example, the inhibitor may be an aryl thiosemicarbazone, such as 1,1′-biphenyl-4-carbaldehyde thiosemicarbazone (compound 1), 4-isopropylbenzaldehyde thiosemicarbazone (compound 2; see, e.g., U.S. Pat. No. 3,849,575), 4-cyclohexylbenzaldehyde thiosemicarbazone (compound 3), or 4-isopropyl-3-nitrobenzaldehyde thiosemicarbazone (compound 4; see, e.g., Saripinar et al. (1996) Arzneimittel-Forschung 46(II):824-8), as described in EXAMPLE 9. Effective amounts and useful routes of administration are described above.

Some embodiments of this aspect of the invention provide methods of treating a subject with a cellular hyperproliferation disorder by administering therapeutically effective amounts of a known therapeutic agent and an inhibitor of the activity of a target protein to the subject, wherein the target protein comprising a sequence that has more than 80% sequence identity to the sequence provided in SEQ ID NO:2 or SEQ ID NO:3. As used herein, the term “known therapeutic agent” includes, but is not limited to, anti-cancer agents and radiation therapy. Thus, the target protein inhibitors of the invention, such as the inhibitors described above, may be administered in combination with known anti-cancer agents. Examples of such agents can be found in Cancer Principles and Practice of Oncology; (Devita & Hellman, eds.), 6^{th} ed. (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, agents that interfere with cell cycle checkpoints. HIV protease inhibitors, reverse transcriptase inhibitors, and other angiogenesis inhibitors.

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate. “Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, and N-4-carboxyphenyl retinamide.

“Cytotoxic/cytostatic agents” refer to compounds which cause cell death or inhibit cell proliferation primarily by interfering directly with the cell's functioning or inhibit or interfere with cell myosis, including alkylating agents, tumor necrosis factors, intercalators, hypoxia activatable compounds, microtubule inhibitors/microtubule-stabilizing agents, inhibitors of mitotic kinesins, inhibitors of kinases involved in mitotic progression, antimetabolites; biological response modifiers; hormonal/anti-hormonal therapeutic agents, haematopoietic growth factors, monoclonal antibody targeted therapeutic agents, topoisomerase inhibitors, proteosome inhibitors and ubiquitin ligase inhibitors.

Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro (2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum(II)]tetrachloride, diarizidinylspermine, arsenic trioxide, 1-(1-dodecylamino-10-hydroxyundecyl)-3,7-dimethylxanthine, zorubicin, idarubicin, daunorubicin, bisantrene, mitoxantrone, pirarubicin, pinafide, valrubicin, anirubicin, antineoplaston, 3′-deamino-3′-morpholino-13-deoxo-10-hydroxycarminomycin, annamycin, galarubicin, elinafide, MEN10755, and 4-demethoxy-3-deamino-3-aziridinyl-4-methylsulphonyl-daunorubicin (see WO 00/50032).

An example of a hypoxia activatable compound is tirapazamine.

Examples of proteosome inhibitors include, but are not limited to, lactacystin and MLN-341 (Velcade).

Examples of microtubule inhibitors/microtubule-stabilising agents include, but are not limited to, paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797. In some embodiments, the epothilones are not included in the microtubule inhibitors/microtubule-stabilising agents.

Examples of topoisomerase inhibitors include, but are not limited to, topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine4-carboxamide, 6-[[2-(dimethylamino)ethyl]amino]-3-hydroxy-7H-indeno[2,1-c]quinolin-7-one, and dimesna.

Examples of inhibitors of mitotic kinesins, and in particular the human mitotic kinesin KSP, are described in PCT Publications WO 01/30768 and WO 01/98278, and pending U.S. Ser. Nos. 60/338,779 (filed Dec. 6, 2001), 60/338,344 (filed Dec. 6, 2001), 60/338,383 (filed Dec. 6, 2001), 60/338,380 (filed Dec. 6, 2001), 60/338,379 (filed Dec. 6, 2001) and WO 03/39460. In an embodiment inhibitors of mitotic kinesins include, but are not limited to inhibitors of KSP, inhibitors of MKLP1, inhibitors of CENP-E, inhibitors of MCAK and inhibitors of Rab6-KIFL.

“Inhibitors of kinases involved in mitotic progression” include, but are not limited to, inhibitors of aurora kinase, inhibitors of Polo-like kinases (PLK) (in particular inhibitors of PLK-1), inhibitors of bub-1 and inhibitors of bub-R1.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine, 3-aminopyridine-2-carboxaldehyde thiosemicarbazone and trastuzumab.

Examples of monoclonal antibody targeted therapeutic agents include those therapeutic agents which have cytotoxic agents or radioisotopes attached to a cancer cell specific or target cell specific monoclonal antibody. Examples include Bexxar. “HMG-CoA reductase inhibitors” refers to inhibitors of 3-hydroxy-3-methylglutaryl-CoA reductase. Compounds which have inhibitory activity for HMG-CoA reductase can be readily identified by using assays well-known in the art. For example, see the assays described or cited in U.S. Pat. No. 4,231,938, at column 6, and WO 84/02131, at pages 30-33. The terms “HMG-COA reductase inhibitor” and “inhibitor of HMG-CoA reductase” have the same meaning when used herein.

Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896), atorvastatin (LIPITOR®; see U.S. Pat. Nos. 5,273,995, 4,681,893, 5,489,691 and 5,342,952) and cerivastatin (also known as rivastatin and BAYCHOL®; see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to fonn the free acid) as well as salt and ester forms of compounds which have HMG-CoA reductase inhibitory activity, and therefor the use of such salts, esters, open-acid and lactone forms is included within the scope of this invention. An illustration of the lactone portion and its corresponding open-acid form is shown below as structures I and II.

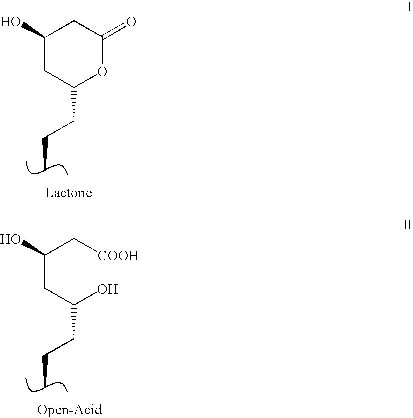

In HMG-CoA reductase inhibitors where an open-acid form can exist, salt and ester forms may be formed from the open-acid, and all such forms are included within the meaning of the term “HMG-CoA reductase inhibitor” as used herein. In some embodiments, the HMG-CoA reductase inhibitor is selected from lovastatin and simvastatin, and in further embodiments, simvastatin. Herein, the term “pharmaceutically acceptable salts” with respect to the HMG-CoA reductase inhibitor shall mean non-toxic salts of the compounds employed in this invention which are generally prepared by reacting the free acid with a suitable organic or inorganic base, particularly those formed from cations such as sodium, potassium, aluminum, calciwn, lithium, magnesium, zinc and tetrainethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenz-imidazole, diethylamine, piperazine, and tris(hydroxymethyl) aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fiumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Ester derivatives of the described HMG-CoA reductase inhibitor compounds may act as prodrugs which, when absorbed into the bloodstream of a warm-blooded animal, may cleave in such a manner as to release the drug form and permit the drug to afford improved therapeutic efficacy.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase). Examples of prenyl-protein transferase inhibiting compounds include (+)-6-[amino(4-chlorophenyl)(1-methyl-1H-imidazol-5-yl)methyl]-4-(3-chlorophenyl)-1-methyl-2(1H)-quinolinone, (−)-6-[amino(4-chlorophenyl)(1-methyl-1H-imidazol-5-yl)methyl]-4-(3-chlorophenyl)-1-methyl-2(1H)-quinolinone, (+)-6-[amino(4-chlorophenyl)(1-methyl-1H-imidazol-5-yl)methyl]-4-(3-chlorophenyl)-1-methyl-2(1H)-quinolinone, 5(S)-n-butyl-1-(2,3-dimethylphenyl)-4-[1-(4-cyanobenzyl)-5-imidazolylmethyl]-2-piperazinone, (S)-1-(3-chlorophenyl)-4-[1-(4-cyanobenzyl)-5-imidazolylmethyl]-5-(2-(ethanesulfonyl)methyl)-2-piperazinone, 5(S)-n-Butyl-1-(2-methylphenyl)-4-[1-(4-cyanobenzyl)-5-imidazolylmethyl]-2-piperazinone, 1-(3-chlorophenyl)-4-[1-(4-cyanobenzyl)-2-methyl-5-imidazolylmethyl]-2-piperazinone, 1-(2,2-diphenylethyl)-3-[N-(1-(4-cyanobenzyl)-1H-imidazol-5-ylethyl)carbamoyl]piperidine, 4-{5-[4-hydroxymethyl-4-(4-chloropyridin-2-ylmethyl)-piperidine-1-ylmethyl]-2-methylimidazol-1-ylmethyl}benzonitrile, 4-{5-[4-hydroxymethyl-4-(3-chlorobenzyl)-piperidine-1-ylmethyl]-2-methylimidazol-1-ylmethyl}benzonitrile, 4-{3-[4-(2-oxo-2H-pyridin-1-yl)benzyl]-3H-imidazol-4-ylmethyl}benzonitrile, 4-{3-[4-(5-chloro-2-oxo-2H-[1,2′]bipyridin-5′-ylmethyl]-3H-imidazol4-ylmethyl}benzonitrile, 4-{3-[4-(2-oxo-2H-[1,2′]bipyridin-5′-ylmethyl]-3H-imidazol-4-ylmethyl}benzonitrile, 4-{3-(2-oxo-1-phenyl-1,2-dihydropyridin-4-ylmethyl)-3H-imidazol-4-ylmethyl}benzonitrile, 18,19-dihydro-19-oxo-5H,17H-6,10:12,16-dimetheno-1H-imidazo[4,3-c][1,11,4]dioxaazacyclo-nonadecine-9-carbonitrile, (±)-19,20-dihydro-19-oxo-5H-18,21-ethano-12,14-etheno-6,10-metheno-22H-benzo[d]imidazo[4,3-k][1,6,9,12]oxatriaza-cyclooctadecine-9-carbonitrile, 19,20-dihydro-19-oxo-5H, 17H-18,21-ethano-6,10:12,16-dimetheno-22H-imidazo[3,4-h][1,8,11,14]oxatriazacycloeicosine-9-carbonitrile, and (±)-19,20-dihydro-3-methyl-19-oxo-5H-18,21-ethano-12,14-etheno-6,10-metheno-22H-benzo [d]imidazo[4,3-k][1,6,9,12]oxa-triazacyclooctadecine-9-carbonitrile.

Other examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359.

For an example of the role of a prenyl-protein transferase inhibitor on angiogenesis see Eur. J. of Cancer 35(9):1394-1401 (1999).

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFRI) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflanunatories (NSAIDs) like aspirin and ibuprofen as well as selective cyclooxy-genase-2 inhibitors like celecoxib and rofecoxib (Proc. Natl. Acad. Sci. U.S.A. 89:7384 (1992); J. Natl. Cancer. Inst. 69:475 (1982); Arch. Opthalmol. 108:573 (1990); Anat. Rec. 238:68 (1994); FEBS Lett. 372:83 (1995); Clin, Orthop. 313:76 (1995); J. Mol. Endocrinol. 16:107 (1996); Jpn. J. Pharmacol. 75:105 (1997); Cancer Res. 57:1625 (1997); Cell 93:705 (1998); Intl. J. Mol. Med. 2:715 (1998); J. Biol. Chem. 274:9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al. (1985) J. Lab. Clin. Med. 105:141-5), and antibodies to VEGF (see Nature Biotechnol. 17:963-8 (1999); Kim et al. (1993) Nature 362:841-4; WO 00/44777; and WO 00/61186).

Other therapeutic agents that modulate or inhibit angiogenesis and may also be used in combination with target protein inhibitors include agents that modulate or inhibit the coagulation and fibrinolysis systems (see review in Clin. Chem. La. Med. 38:679-92 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-54 (2001)). TAFIa inhibitors have been described in WO 03/13526 and U.S. Ser. No. 60/349,925 (filed Jan. 18, 2002).

“Agents that interfere with cell cycle checkpoints” refer to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

“Inhibitors of cell proliferation and survival signalling pathway” refer to compounds that inhibit signal transduction cascades downstream of cell surface receptors. Such agents include inhibitors of serine/threonine kinases, including but not limited to inhibitors of Akt such as described in WO 02/083064, WO 02/083139, WO 02/083140 and WO 02/083138), inhibitors of Raf kinase (for example BAY-43-9006), inhibitors of MEK (for example CI-1040 and PD-098059), inhibitors of mTOR (for example Wyeth CCI-779), and inhibitors of P13K (for example LY294002).

The combinations with NSAID's are directed to the use of NSAIDs which are potent COX-2 inhibiting agents. For purposes of this specification an NSAID is potent if it possess an IC₅₀ for the inhibition of COX-2 of 1 micromolar or less as measured by cell or microsomal assays.

The invention also encompasses combinations with NSAIDs which are selective COX-2 inhibitors. For purposes of this specification NSAIDs which are selective inhibitors of COX-2 are defined as those which possess a specificity for inhibiting COX-2 over COX-1 of at least 100 fold as measured by the ratio of IC₅₀ for COX-2 over IC₅₀ for COX-1 evaluated by cell or microsomal assays. Such compounds include, but are not limited to those disclosed in U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, U.S. Pat. No. 5,710,140, issued Jan. 20, 1998, WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999, all of which are hereby incorporated by reference.

Inhibitors of COX-2 that are useful in the instant method of treatment include:
3-phenyl-4-(4-(methylsulfonyl)phenyl)-2-(5H)-furanone; and 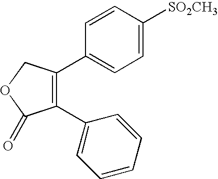
5-chloro-3-(4-methylsulfonyl)phenyl-2-(2-methyl-5-pyridinyl)pyridine; 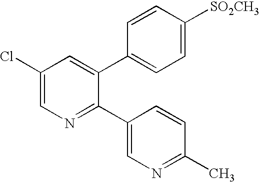
or a pharmaceutically acceptable salt thereof.

General and specific synthetic procedures for the preparation of the COX-2 inhibitor compounds described above are found in U.S. Pat. No. 5,474,995, issued Dec. 12, 1995, U.S. Pat. No. 5,861,419, issued Jan. 19, 1999, and U.S. Pat. No. 6,001,843, issued Dec. 14, 1999, all of which are herein incorporated by reference.

Compounds that have been described as specific inhibitors of COX-2 and are therefore useful in the present invention include, but are not limited to, the following: 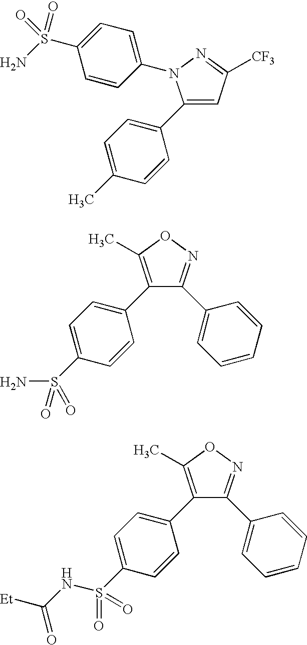
or a pharmaceutically acceptable salt thereof.

Compounds that are described as specific inhibitors of COX-2 and are therefore useful in the present invention, and methods of synthesis thereof, can be found in the following patents, pending applications and publications, which are herein incorporated by reference: WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999.

Compounds that are specific inhibitors of COX-2 and are therefore useful in the present invention, and methods of synthesis thereof, can be found in the following patents, pending applications and publications, which are herein incorporated by reference: U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, and U.S. Pat. No. 5,710,140, issued Jan. 20, 1998.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RP14610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αᵥβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some specific examples of tyrosine kinase inhibitors include N-(trifluoromethylphenyl)-5-methylisoxazol-4-carboxamide, 3-[(2,4-dimethylpyrrol-5-yl) methylidenyl]indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, ST1571A, N-4-chlorophenyl4-(4-pyridylmethyl)-1-phthalazinamine, and EMD 121974.

Combinations with compounds other than anti-cancer compounds are also encompassed in the methods of the invention. For example, combinations of the instantly claimed compounds with PPAR-γ (i.e., PPAR-gamma) agonists and PPAR-δ (i.e., PPAR-delta) agonists are useful in the treatment of certain malingnancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. (1998) 31:909-13; J. Biol. Chem. (1999) 274:9116-21; Invest. Ophthalmol Vis. Sci. (2000)41:2309-17). More recently, PPAR-γ agonists have been shown to inhibit the angiogenic response to VEGF in vitro; both troglitazone and rosiglitazone maleate inhibit the development of retinal neovascularization in mice (Arch. Ophthalmol. (2001) 119:709-17). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, GI262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy)phenoxy)propoxy)-2-ethylchromane-2-carboxylic acid (disclosed in U.S. Ser. Nos. 60/235,708 and 60/244,697).

In some embodiments of the invention, target protein inhibitors are used in combination with gene therapy for the treatment of cancer. For an overview of genetic strategies to treating cancer see Hall et al. (1997) Am J Hum Genet 61:785-9. and Kufe et al. (2000) Cancer Medicine, 5th ed, pp 876-89, BC Decker, Hamilton). Gene therapy can be used to deliver any tumor suppressing gene. Examples of such genes include, but are not limited to, p53, which can be delivered via recombinant virus-mediated gene transfer (see, e.g., U.S. Pat. No. 6,069,134), a uPA/uPAR antagonist (“Adenovirus-Mediated Delivery of a uPA/uPAR Antagonist Suppresses Angiogenesis-Dependent Tumor Growth and Dissemination in Mice,” Gene Therapy 5(8):1105-13 (1998)), and interferon gamrnma (J Immunol. 164:217-22 (2000)).

Target protein inhibitors may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

Target protein inhibitors may be employed in conjunction with anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. For the treatment or prevention of emesis that may result upon administration of the target protein inhibitors, conjunctive therapy with an anti-emesis agent may be selected from a neurokinin-1 receptor antagonist, a SHT3 receptor antagonist, and a corticosteroid.

Neurokinin-1 receptor antagonists of use in conjunction with the target protein inhibitors of the present invention are fully described, for example, in U.S. Pat. Nos. 5,162,339, 5,232,929, 5,242,930, 5,373,003, 5,387,595, 5,459,270, 5,494,926, 5,496,833, 5,637,699, 5,719,147; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942 and 97/21702; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications, which are incorporated herein by reference.

In some embodiments, the neurokinin-1 receptor antagonist for use in conjunction with the compounds of the present invention is selected from: 2-(R)-(1-(R)-(3,5-bis(trifluoromethyl)phenyl)ethoxy)-3-(S)-(4-fluorophenyl)4-(3-(5-oxo-1 H,4H-1,2,4-triazolo)methyl)morpholine, or a pharmaceutically acceptable salt thereof, which is described in U.S. Pat. No. 5,719,147.

Target protein inhibitors may also be administered with an agent useful in the treatment of anemia. Such an anemia treatment agent is, for example, a continuous eythropoiesis receptor activator (such as epoetin alfa).

Target protein inhibitors may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

Target protein inhibitors may also be administered with an immunologic-enhancing drug, such as levamisole, isoprinosine and Zadaxin.

In a third aspect, the invention provides methods for identifying candidate subjects for treatment with a modulator of the activity of a target protein. The methods comprise the steps of: (a) measuring the level of expression of a target protein in sample cells from a subject, wherein the target protein comprises a sequence that has more than 80% sequence identity to the sequence provided in SEQ ID NO:2 or SEQ ID NO:3; and (b) identifying the subject as a candidate subject for treatment with a modulator of the activity of the target protein if the level of expression of the target protein in the sample cells is significantly different than in control cells.

In the first step, the level of expression of a target protein in sample cells of the subject is measured. As used herein, the term “sample cells” refers to cells from any clinically relevant tissue sample, such as a tumor biopsy or fine needle aspirate, or a sample of bodily fluid, such as blood, plasma, serum, lymph, ascitic fluid, cystic fluid, urine, or nipple exudate. The sample may be taken from a human or from a non-human subject. The target proteins used in the methods of this aspect of the invention are as described above for the methods of the first aspect of the invention.

The level of expression of the target protein may be determined by any means known in the art. The expression level may be determined by isolating and measuring the amount of nucleic acid transcribed from the gene encoding the target protein. Alternatively, or additionally, the amount of target proteins translated may be determined. For example, the level of expression of the target protein may be determined by isolating RNA from the sample and hybridizing it to nucleic acid probes specific for the DNA or RNA equivalent of the transcript of the gene encoding the target protein. Useful techniques for measuring mRNA levels include, but are not limited to, quantitative reverse transcriptase PCR, Northern analysis, RNase protection, and hybridization to microarrays. The level of expression of the target protein may also be assessed at the protein level. Useful techniques for measuring protein levels include, but are not limited to, standard immunoassays using antibodies to the target protein, mass spectroscopy assays, antibody microarrays, and 2D gel electrophoresis assays. Exemplary methods for measuring expression levels of a target protein is provided in EXAMPLES 1, 2, and 6.

In the second step, the subject is identified as a candidate subject for treatment with a modulator of the activity of a target protein if the level of expression of the target protein in the sample cells is significantly different than in control cells. The term “control cells” refers to reference cells that are expressing a desirable level of the target protein. The control cells may be, but are not necessarily, cells from the same subject from which the sample cells are obtained. Control cells may be obtained from the same tissue from which the sample cells are obtained. The control cells may also be cells from the same tissue type but from a different subject. Control cells may also include hypothetical cells, for example, imaginary cells that represent an average of target protein expression levels in multiple subjects or that represent an idealized level of expression of the target protein.

The comparison of the levels of expression in the sample cells and the control cells may be made using conventional methods in the art. For example, comparison of expression levels may be accomplished visually or by means of a densitometer. Generally, methods for comparing levels of gene expression provide an estimate of the statistical significance of the difference in expression levels. For example, repeated measurements of individual samples may be used to estimate the mean and standard error of a measured expression level. According to the methods of this aspect of the invention, if the difference in expression levels in control cells and abnormally proliferating cells is determined to be statistically significant, the subject is identified as a candidate subject for treatment with a modulator of the activity of the target protein. In some embodiments, the methods of this aspect of the invention further comprise treating the candidate subject by administering a modulator of the activity of the target protein, as described above.

In some embodiments, the methods identify candidate subjects for treatment with an inhibitor of the activity of a target protein. However, the methods of this aspect of the invention are also applicable for identifying candidate subjects for treatment with a modulator that stimulates the activity of the target protein. The methods for identifying candidate subjects for treatment with an inhibitor of the activity of a target protein comprise the steps of: (a) measuring the level of expression of the target protein in abnormally proliferating cells of a subject, wherein the target protein comprises a sequence that has more than 80% sequence identity to the sequence provided in SEQ ID NO:2 or SEQ ID NO:3; and (b) identifying the subject as a candidate subject for treatment with inhibitors of the activity of a target protein if the level of expression of the target protein in the abnormally proliferating cells is significantly higher than in control cells. According to this embodiment, the sample cells are abnormally proliferating cells. Typically, the control cells used in this embodiment are cells that are not proliferating abnormally. In some embodiments, the methods of this aspect of the invention further comprise treating the candidate subject by administering an inhibitor of the activity of the target protein, as described above.

The present invention also provides kits for screening for modulators of target proteins comprising a sequence that has more than 80% sequence identity to the sequence provided in SEQ ID NO:2 or SEQ ID NO:3. These kits may contain materials and reagents for screening for modulators of the target proteins and instructions describing how to perform the screen. For example, the kits may include a biologically active target protein, reaction tubes, and instructions for testing the activity of the target protein. The kits may be tailored to the use of a specific assay for the activity of the target protein. Thus, the kits may be tailored for ATPase assays, microtubule-binding assays, microtubule-gliding assays, or cell growth and viability assays.

Examples provided are intended to assist in a further understanding of the invention and illustrate the best mode now contemplated for practicing the invention. Particular materials employed, species and conditions are intended to be illustrative of the invention and not limiting the reasonable scope thereof.

### EXAMPLE 1

This Example describes the KIF14 expression levels in normal and tumor cells.

KIF14 mRNA expression levels were assessed in two normal cell lines, human mammary epithelial cells HMEC (Cambrex Corporation (Clonetics), Cat. No. CC-2551) and human skeletal muscle cells SKMC (Cambrex Corporation, East Rutherford, N.J. (BioWhittaker), Cat. No. CC-2561), and two tumor cell lines, colorectal cancer cell line HT-29 (American Type Culture Collection (ATCC), Cat. No. HTB-38) and breast cancer cell line MCF-7 (ATCC, Cat. No. HTB-22). RNA from these cell lines was harvested by following the standard QIAshredder (Qiagen, Cat. No. 79656) homogenization and Qiagen RNeasy protocol (Qiagen, Cat. No. 74106) with an RNase-free DNase step (Qiagen, Cat. No. 7925). Preparation of labeled copy RNA for hybridization to custom made human hu25k microarrays (Agilent Technologies, Inc., Palo Alto, Calif.), hybridization conditions, and subsequent data processing are as previously described (van't Veer et al. (2002) Nature 415:530-536).

Results: The level of expression of KIF14 mRNA was between about 4 and about 6.6 times higher in the tumor cell lines than in normal cells, as shown in Table 1.

TABLE 1
KIF14 mRNA Expression in Normal and Tumor Cells
Relative KIF14 mRNA Expression Levels

| Cells | Relative-Fold Intensity |
|---|---|
| SKMC | 1.0 |
| HMEC | 1.3 |
| HT-29 | 4.0 |
| MCF7 | 6.6 |

To measure the KIF14 mRNA expression in a panel of different human tissues and tumor cell lines, 29 oligonucleotide probes from locations throughout the KIF14 transcript sequence (SEQ ID NO:1) were generated and placed on a microarray. RNA from 68 tissues/cell lines was amplified using a full-length amplification protocol, labeled with either Cy3 or CyS dyes, and was hybridized to the microarray, as previously described in Hughes et al. (2001) Nature Biotechnol. 19:342-347 and van't Veer et al. (2002) Nature 415:530-536. mRNA expression in each tissue was calculated as the exponential of the average of the probe natural-log intensities, after background subtraction and dye-normalization. Error estimates represent a combination of modeled probe measurement error and the difference between probes. KIF14 was generally found to be expressed at high levels in tumor cell lines, and at lower levels in human tissues, as shown in Table 2.

TABLE 2
KIF14 mRNA Expression in Human
Tissues and Tumor Cell Lines

| Tissue | Exp. | Err. |
|---|---|---|
| Lung carcinoma (A549) | 3903 | 623 |
| Leukemia-chronic myelogenous (K562) | 3126 | 524 |
| Leukemia-lymphoblastic (MOLT-4) | 1971 | 386 |
| Colorectal adenocarcinoma (SW480) | 1860 | 342 |
| Leukemia promyelocytic (HL-60) | 1859 | 303 |
| HeLa | 1676 | 271 |
| Lymphoma-Burkitt's (Daudi) | 1536 | 375 |
| Salivary gland | 1184 | 202 |
| Melanoma (G361) | 1177 | 212 |
| Bone marrow | 920 | 174 |
| Liver-fetal | 875 | 212 |
| Lymphoma-Burkitt's (Raji) | 863 | 138 |
| Testes | 615 | 112 |
| Colon-transverse | 538 | 100 |
| Tonsil | 465 | 82 |
| Colon-descending | 433 | 79 |
| Ileum | 355 | 100 |
| Retina | 352 | 78 |
| Bladder | 333 | 86 |
| Lung-fetal | 306 | 65 |
| Liver-left lobe | 265 | 67 |
| Kidney-fetal | 262 | 66 |
| Duodenum | 260 | 57 |
| Stomach | 252 | 52 |
| Placenta | 234 | 50 |
| Spinal cord-fetal | 207 | 51 |
| Brain-fetal | 200 | 47 |
| Lymph node | 185 | 46 |
| Jejunum | 175 | 56 |
| Ileocecum | 154 | 33 |
| Uterus-corpus | 147 | 46 |
| Uterus | 135 | 37 |
| Spleen | 133 | 31 |
| Adrenal medulla | 121 | 30 |
| Brain | 104 | 32 |
| Brain-corpus callosum | 102 | 31 |
| Kidney | 102 | 29 |
| Thyroid | 100 | 28 |
| Brain-postcentral gyrus | 98 | 35 |
| Trachea | 98 | 27 |
| Liver | 94 | 25 |
| Lung | 91 | 32 |
| Spinal cord | 91 | 27 |
| Brain-nucleus accumbens | 88 | 33 |
| Epididymus | 85 | 26 |
| Adrenal cortex | 84 | 23 |
| Brain-amygdala | 83 | 31 |
| Thymus-normal | 77 | 25 |
| Brain-cerebellum | 76 | 24 |
| Brain-hippocampus | 75 | 29 |
| Brain-thalamus | 72 | 23 |
| Prostate | 68 | 27 |
| Pancreas | 66 | 29 |
| Brain-caudate nucleus | 62 | 28 |
| Brain-cerebral cortex | 51 | 27 |
| Tongue | 50 | 25 |
| Heart | 35 | 15 |
| Skeletal muscle | 35 | 12 |
| Brain-medulla oblongata | 31 | 19 |
| Brain-paracentral gyrus | 30 | 17 |
| Adrenal gland | 28 | 15 |
| Adipose tissue | 24 | 12 |
| Lung-upper right lobe | 13 | 8 |
| Brain-hypothalamus | 12 | 8 |
| Brain-frontal lobe | 10 | 5 |
| Brain-temporal lobe | 8 | 5 |
| Brain-putamen | 7 | 5 |
| Dorsal root ganglion | 4 | 3 |

Exp. = Expression
Err. = Error Estimate

### EXAMPLE 2

This Example describes the similarity of the KIF14 mRNA expression pattern in cell lines treated with growth factors to the mRNA expression pattern of mitotic kinesins.

Cell lines MCF-7, HT-29, SKMC, and HMEC, described in EXAMPLE 1, were used. Fifty-six 10 cm plates were seeded with the each cell line to given a density of 70-80% confluence on the first day of the experiment. The cells were serum starved by the aspiration of the 10% FBS/DMEM and the addition of 0.2% FBS/DMEM (charcoal stripped serum). After 24 hours of serum starvation at 37° C., 5 sets of 5 plates were treated with 100 ng/mL of EGF (Upstate Biotechnology, Cat. No. 01-407), 100 ng/mL of β-FGF (Promega, Cat. No. G507A), 100 ng/mL of IGF-1 (Sigma, Cat. No. I3769), 100 ng/mL of insulin (Sigma, Cat. No. 12767), and 30 ng/mL of heregulin (NeoMarkers, Cat. No. RP-318-P1AX). Growth factors were resuspended (where applicable) and stored according to the manufacturers instructions. Five sets of 5 plates were correspondingly treated with 0.2% FBS/DMEM (charcoal stripped serum) as a control solution. An additional 6 plates were treated with one of the 5 growth factors or the control. Control plates were done in tandem with their matched treated sample. These latter 6 plates were lysed after 15 minutes for standard Western blotting of phosphorylated Akt and MAPK to verify that the stimulation had occurred. The remaining plates were incubated for a fixed amount of time in treated and control pairs (30 minutes, 2 hours, 6 hours, 18 hours, and 24 hours) prior to harvesting the RNA following the standard QIAshredder (Qiagen, Cat. No. 79656) homogenization and Qiagen RNeasy protocol (Qiagen, Cat. No. 74106) with an RNase-free DNase step (Qiagen, Cat. No. 7925). Preparation of labeled copy RNA for hybridization to custom made human hu25k microarrays (Agilent Technologies, Inc., Palo Alto, Calif.), hybridization conditions, and subsequent data processing are as previously described (van't Veer et al. (2002) Nature 415:530-536).

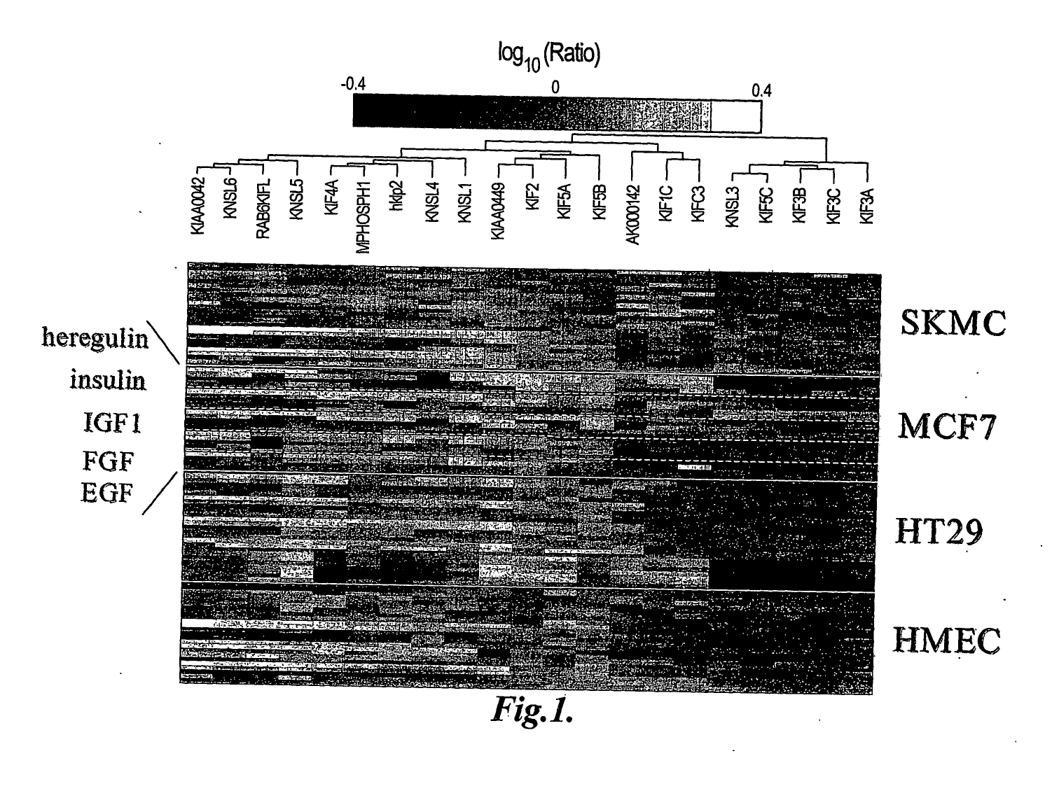

Results: The mRNA expression pattern in cell lines treated with growth factors was different from that of neuronal kinesins but similar to that of nine known mitotic cyclins, CENP-E, KIF4A, MPOHOPH1, hklp2, KNSL6, RAB6KIFL, KNSL5, KNSL4, and KNSL1, as shown in FIG. 1.

### EXAMPLE 3

This Example describes the accumulation of KIF14 mRNA and the dynamic cellular localization of KIF14 protein during mitosis.

Transcript accumulation during mitosis is a defining characteristic of mitotic kinesins (Yen et al. (1992) Nature 359(6395):53609; Hill et al. (2000) EMBO J. 19(21):5711-9). In addition, cellular localization studies have been instrumental in elucidating the function of mitotic cyclins (Yen et al. (1992) Nature 359(6395):53609; Hill et al. (2000) EMBO J. 19(21):5711-9; Matuliene et al. (2002) Mol. Biol. Cell. 13(6):1832-45; Abaza et al. (2003) J. Biol. Chem. 278(3):27844-52). Immunofluorescence microscopy was used to visualize the localization of KIF14 protein throughout the cell cycle and microarray profiling was used to analyze the accumulation of KIF14 mRNA in synchronized cells.

Thymidine cell synchronization: HCT116 cells were seeded in 10 cm plates at 1.5×10⁶ cells per plate and grown overnight. The original media was aspirated and 10 mls of fresh, filtered media containing 2 mM thymidine (Sigma, cat. no. T-1895, lot No. 28H0393) was added to each plate in order to block cells at G1/S. Cells were incubated at 37° C. for 15-16 hrs. Thymidine containing media was aspirated and cells were released from the thymidine block with 2×5 ml washes in PBS followed by addition of 10 mls of media containing 24 microM deoxycytidine. Cells were incubated at 37° C. for 10 hrs followed by media aspiration, a 5 ml PBS wash and repeat of the G1/S block by addition of 10 mls of 2 mM thymidine containing media. After 15 hrs of thymidine block, the cells were washed and put into deoxycytidine media which was then t=0 for the time course. Samples were collected for FACS, and RNA extraction at 2 hr intervals from t=0 to 24 hrs with one additional point at 36 hrs.

Molecular profiling of synchronized HCT116 cells: For each 10 cm plate, at each designated timepoint, the culture media was completely aspirated and cells were lysed in RLT buffer (Qiagen, Inc. (Valencia, Calif.), RNeasy kit) containing 1% BME. Cells were scraped and the lysate pipeted to mix and reduce clumps. Cell lysates were homogenized using QIA shredder spin columns and total cellular RNA was isolated using the Rneasy mini kit (Qiagen). RNA amplification, labeling, and hybridization to hu25K ink-jet DNA microarrays was carried out as previously described (Hughes et al. (2001) Nat. Biotechnol. 19:342-7; van't Veer et al. (2002) Nature 415:530-6).

KIF14 transcripts accumulated in cells progressing through G2/M. The cell cycle dependent regulation of KIF14 expression mirrored that of the known mitotic kinesin, CENPE. These observations lend strong support to the hypothesis that KIF14 fimctions as a mitotic kinesin.

Immunofluorescence Microscopy: HeLa-S3 cells cultured on glass chamber well slides were fixed and permeabilized for 15 minutes in immunohistochemical buffer containing 100 mM PIPES (pH 6.8), 10 mM EGTA, 1 mM MgCl₂, 0.2% Triton X-100, 4% formaldehyde (Kapoor et al. (2000) J. Cell. Biol. 150(5):975-88). Following fixation, cells were washed 2 times with TBST (see EXAMPLE 5) and incubated with primary antibody for 2 hours at 37° C. Rabbit anti-KIF14 polyclonal antibody was obtained from Abcam, Inc. (ab3746) and used at a 1:500 dilution. Anti-alpha tubulin monoclonal antibody (clone DM1A0) was obtained from SIGMA and used at a 1:500 dilution. Cells were washed 2 times in TBST and primary antibody binding was detected using Alexa Fluor 488 goat anti-rabbit IgG (Molecular Probes) and Alexa Fluor 594 goat anti-mouse IgG (Molecular Probes) both used at a 1:250 dilution. Incubation with secondary antibodies was carried out at room temperature for 1 hour followed by 3 washes in TBST supplemented with 10 micrograms/ml Hoechst in order to stain DNA. Slides were mounted with Fluoromount G (Southern Biotech) and visualized directly on a Deltavision v3.5 deconvolution microscope (Applied Precision, Inc.) using DAPI, FITC, and RD-TR-PE filter sets for blue, green, and red, respectively.

KIF14 was dispersed diffusely within the cytoplasm in interphase cells, but in prophase cells it was localized to the centrosomes and their associated microtubules. In metaphase cells, KIF14 was located at the spindle poles and along spindle microtubules. In anaphase cells, KIF14 was found in the spindle midzone, whereas in telophase cells it was more concentrated and co-localized with the midbody matrix and contractile ring. KIF14 was also found localized in extracellular ring-like structures associated with tubulin, reminiscent of contractile rings after completion of cellular abscission. The subcellular localization of KIF14 suggests a role for this protein in cytokinesis. In contrast, KLP38B, the Drosophila KIF14 ortholog (Molina et al. (1997) J. Cell. Biol. 139(6):1361-71), co-localized with condensed chromatin, suggesting it functions during chromosome segregation.

### EXAMPLE 4

This Example describes that transfection of KIF14 siRNA results in the cell growth inhibition and cell death.

Transfection of siRNA: siRNA transfection was used to lower (knockdown) the levels of KIF14 mRNA in order to determine the loss-of-function phenotype of KIF14. One day prior to transfection, 100 microliters of cervical cancer HeLa cells (ATCC, Cat. No. CCL-2), colorectal cancer HCTI 16 cells (ATCC, Cat. No. CCL-247), or melanoma A2058 cells (ATCC, Cat. No. CRL-1147) grown in DMEM/10% fetal bovine serum (Invitrogen, Carlsbad, Calif.) to approximately 90% confluency were seeded in a 96-well tissue culture plate (Corning, Corning, N.Y.) at 1500 cells/well. For each transfection 85 microliters of OptiMEM® (Invitrogen) was mixed with 5 microliter of siRNA (Dharnacon, Denver) from a 20 micromolar stock. Two KIF14 siRNA sequences were used:

| | |
|---|---|
| KIF14-4476: | |
| 5′ AAACUGGGAGGCUACUUACdTdT 3′; | (SEQ ID NO:8) |
| and | |
| KIF14-5128: | |
| 5′ CUCACAUUGUCCACCAGGAdTdT 3′. | (SEQ ID NO:9) |

As a control, luciferase siRNA (5′CGUACGCGGAAUACUUCGAdTdT 3′, SEQ ID NO:10) was transfected into each of the three different cell lines. For each transfection 5 microliter OptiMEM® was mixed with 5 microliter Oligofectamine™ reagent (Invitrogen) and incubated 5 minutes at room temperature. The 10 microliter OptiMEM®/Oligofectamine™ mixture was dispensed into each tube with the OptiMEM®/siRNA mixture, mixed and incubated 15-20 minutes at room temperature. 10 microliter of the transfection mixture was aliquoted into each well of the 96-well plate and incubated for 4 hours at 37° C. and 5% CO₂. After 4 hours, 100 microliter/well of DMEM/10% fetal bovine serum was added and the plates were incubated at 37° C. and 5% CO₂ for 72 hours.

alamarBlue™ Assay for Cell Growth: The alamarBlue™ assay is a measure of cellular respiration and is used as a measure of live cell number. The internal environment of the proliferating cell is more reduced than that of non-proliferating cells. Specifically, the ratios of NADPH/NADP, FADH/FAD, FMNH/FMN, and NADH/NAF increase during proliferation. alamarBlue™ can be reduced by these metabolic intermediates and, therefore, can be used to monitor cell proliferation.

72 hours after transfection with siRNAs, the alamarBlue™ assay was performed to determine whether KIF14 siRNA transfection results in reduced cell growth and/or increased cell death. 72 hours after transfection the medium was removed from the wells and replaced with 100 microliter/well DMEM/10% Fetal Bovine Serum (Invitrogen) containing 10% (vol/vol) alamarBlue™ reagent (Biosource International Inc., Camarillo, Calif.) and 0.001 volumes of 1M Hepes buffer tissue culture reagent (Invitrogen). The plates were incubated 2 hours at 37° C. and the plate was read at 570 and 600 nm wavelengths on the SpectraMax plus plate reader (Molecular Devices, Sunnyvale, Calif.) using Softmax Pro 3.1.2 software (Molecular Devices).

The alamarBlue™ reduction was calculated as percent reduced using the equation:

$$Percent Reduced = \frac{( {ɛ}_{ox} {λ}_{2} ) ( A {λ}_{1} ) - ( {ɛ}_{ox} {λ}_{1} ) ( A {λ}_{2} ) \times 100}{( {ɛ}_{red} {λ}_{1} ) ( {A}^{′} {λ}_{2} ) - ( {ɛ}_{red} {λ}_{2} ) ( {A}^{′} {λ}_{1} )}$$

where:
λ₁=570 nm
λ₂=600 nm
(ε_{red} λ₁)=155,677 (Molar extinction coefficient of reduced alamarBlue™ at 570 nm)
(ε_{red} λ₂)=14,652 (Molar extinction coefficient of reduced alamarBlue™ at 600 nm)
(εₒₓ λ₁)=80,586 (Molar extinction coefficient of oxidized alamarBlue™ at 570 nm)
(εₒₓ λ₂)=117,216 (Molar extinction coefficient of oxidized alamarBlue™ at 600 nm)
(A λ₁)=Absorbance of test wells at 570 nm
(A λ₂)=Absorbance of test wells at 600 nm
(A′λ₁)=Absorbance of negative control wells which contain medium plus alamarBlue™, but to which no cells have been added at 570 nm.
(A′λ₂)=Absorbance of negative control wells which contain medium plus alamarBlue™ but to which no cells have been added at 600 nm.

The percent reduced of the wells containing no medium was subtracted from the percent reduced of the wells containing samples to determine the percent reduced above background. The percent reduced for wells transfected with KIF14 siRNAs were compared to luciferase siRNA-transfected wells. The number calculated for percent reduced for luciferase siRNA-transfected wells was considered to be 100%.

Caspase Activity Measurement: Caspase activity is an indicator of cell death. To measure caspase activity in control and KIF14 siRNA-transfected cells, HeLa cells were seeded at a density of 2000 cells/well in a 96-well black wall clear bottomed tissue culture treated Costar Plate (Costar, Cat. No. 3603) in 100 microliters of DMEM+10% FBS (with no antibiotics added). All 37° C. incubations were performed in a humidified 5% CO₂ incubator. After overnight incubation at 37° C., each well was treated with a transfection mixture to generate a final concentration of 100 nM oligo duplex (Dharmacon, A4 preparation), with 0.5 microliter Oligofectamine™ (Invitrogen, Cat. No. 12252-011), in a total of 10 microliters of Optimem®, which had been allowed to sit 15-20 minutes at ambient temperature prior to addition. After 4-16 hours at 37° C., an additional 90 microliters of warmed media was added. Cells were incubated with the transfection mixture at 37° C. for a total of 48 hours. The plates were then spun in a Beckman centrifuge (rotor JS-4.2) at 1200 rpm for 10 minutes at 4° C. prior to the aspiration of the media and addition of 40 microliters of lysis buffer (from the ApoAlert kit, Clonetech—Fluorescent detection Caspase 3 kit, Cat. No. K2026-2) to each well. The plates were incubated for 20 minutes at 4° C. prior to the addition of 10 microliters of a substrate reaction stock solution. This stock is a solution of 20 microliters of 10 mM DEVD-afc (from Biosource, Cat. No. 77-935, 25 mg,—add DMSO to 10 mM), 20 microliters of 1 M DTT (final concentration about 6 mM), and 760 microliters of 5× Buffer. The 5× Buffer is composed of 250 mM Tris-HCl, 50 mM NaCl, 5 mM MgCl₂, 5 mM DTT, 5 mM EDTA, and 25% Glycerol. The plates were covered with adhesive foil and incubated overnight at 37° C. Plates were read on a Gemini SpectroMax at excitation 400 nm, emission 505 nm.

Results: There was significant growth inhibition of all three cell types after transfection of either of the two KIF14 siRNA, compared to luciferase siRNA-transfected control cells as shown in Table 3.

TABLE 3
Growth Inhibition by KIF14 siRNA

| | Mean | Standard |
|---|---|---|
| siRNA Cells | (%) | Deviation |
| Luciferase siRNA (SEQ ID NO: 10) | 100 | |
| Control Cells | | |
| KIF 4476 (SEQ ID NO: 8) | 26.0 | 2.4 |
| HeLa Cells | | |
| KIF 5128 (SEQ ID NO: 9) | 39.4 | 6.7 |
| HeLa Cells | | |
| KIF 4476 (SEQ ID NO: 8) | 45.5 | 7.4 |
| HCT116 Cells | | |
| KIF 5128 (SEQ ID NO: 9) | 61.7 | 1.9 |
| HCT116 Cells | | |
| KIF 4478 (SEQ ID NO: 8) | 33.9 | 8.9 |
| A2058 Cells | | |
| KIF 5128 (SEQ ID NO: 9) | 61.7 | 10.6 |
| A2058 Cells | | |

There was a significant induction of caspase activity in KIF siRNA-treated HeLa cells compared to luciferase siRNA-treated HeLa cells, as shown in Table 4. The relative amount of caspase activity in KIF siRNA-treated HeLa cells suggests that the growth inhibition observed in the alamarBlue™ assay is due, at least in part, to cell death.

TABLE 4
Induction of Caspase Activity by KIF siRNA

| | | Standard |
|---|---|---|
| siRNA/HeLa Cells | Mean | Deviation |
| Luciferase/Control Cells | 1.0 | 0.005 |
| KIF14-4476 (SEQ ID NO: 8) | 5.7 | 0.4 |
| KIF14-5128 (SEQ ID NO: 9) | 2.1 | 0.2 |

### EXAMPLE 5

This Example describes that transfection of KIF14 siRNA results in aberrant cytokinesis and/or apoptosis in HeLa cells.

Cell Morphology Immunofluorescence Methods: HeLa cells were seeded at a density of 2000 cells/well in a 96-well black wall clear bottomed tissue culture treated Costar Plate (Costar Cat, Cat. No. 3603) in 100 microliters of DMEM+10% FBS (with no antibiotics added). After overnight incubation at 37° C., each well was treated with a transfection mixture to generate a final concentration of 100 nM oligo duplex (Dharmacon, A4 preparation), 0.5 microliter Oligofectamine™ (Invitrogen, Cat No. 12252-011), in a total of 10 microliters Optimem®, which had been allowed to sit for 15-20 minutes at ambient temperature prior to addition. As a control, HeLa cells were mock-transfected with Oligofectamine™ alone. After 4-16 h at 37° C., an additional 90 microliters of warmed media was added. Cells were incubated for a designated number of hours (24, 48 or 72 h) with the transfection mixture at 37° C. The media was aspirated from the plates prior to their submersion in a vat of −20° C. methanol. After 10 minutes at −20° C., the methanol was removed and 100 microliters of a pre-made mixture of the reagents was added. This mixture was composed of 10 ml TBST (TBS containing 0.2% Triton X-100 (Sigma, Cat. No. T 9284) 5 mg/ml BSA (Roche, Cat. No. 100377), and 0.05% NaN3), 1:1000 DM1A monoclonal antibody (mouse IgG1, Sigma, Cat. No. T9026), 1:1000 Alexa-Fluor® 488 (Molecular Probes, Cat. No. A-11029 goat anti-mouse at 488 nm), and 10 microliters of a 1 mg/mL stock of DAPI stain (Sigma, Cat. No. D 9542). The plates were then stored at 4° C. for 1-2 hours prior to being rinsed with TBST and imaged.

Procedure for Tabulation of Imaging Data from the KIF14 siRNA Time Course: HeLa cells were counted manually from individual 40× fields obtained from the 96-well plates stained according to the procedure for immunofluorescence. Each cell was categorized as being in: (1) cytokinesis, (2) other phases of a mitosis of normal appearance, (3) other phases of mitoses of abnormal appearance (tripartite metaphases, asters or partial asters, lagging chromosomes, etc.), (4) interphase with two nuclei (binucleate), (5) interphase with greater than two nuclei (multinucleate), (6) apoptosis (characterized by blebbing and/or rounding and condensed chromatin), or (7) other. No cell was counted more than once for the above analysis. The total number of cells per field was also assessed, counting binucleate, multinucleate, or cells still in the process of cytokinesis as single cells.

Results: Table 5 shows the percentage of total cells in cytokinesis, the percentage of cells in normal mitoses, the percentage of cells in abnormal mitoses, the percentage of binucleate cells, the percentage of multinucleate cells, and the percentage of apoptotic cells. These results suggest that the KIF14 may be involved in cytokinesis, and that reduction of KIF14 expression using RNA interference may result in aberrant cytokinesis, the formation of binucleate cells, and apoptosis.

TABLE 5
Data from KIF14 siRNA Time Course

| | Control | | | | | | |
|---|---|---|---|---|---|---|---|
| | Average | KIF14-4476 | | | KIF14-5128 | | |
| | of 3 time | (SEQ ID NO: 8) | | | (SEQ ID NO: 9) | | |
| | points | 24 h | 48 h | 72 h | 24 h | 48 h | 72 h |
| Cytokinesis (%) | 4 | 14 | 8 | 7 | 20 | 14 | 6 |
| Normal Mitosis | 3 | 1 | 2 | 0.9 | 3 | 1 | 2 |
| (%) | | | | | | | |
| Abnormal Mitosis | 3 | 0.3 | 0.6 | 0.9 | 0.7 | 0.7 | 0.5 |
| (%) | | | | | | | |
| Binucleate Cells | 5 | 6 | 7 | 9 | 4 | 10 | 9 |
| (%) | | | | | | | |
| Multinucleate | 1 | 0.3 | 2 | 2 | 1 | 3 | 3 |
| Cells (%) | | | | | | | |
| Apoptotic Cells | 0.3 | 1 | 4 | 13 | 2 | 6 | 13 |
| (%) | | | | | | | |
| Total Cells | 377 | 294 | 531 | 444 | 307 | 430 | 373 |
| Counted | | | | | | | |

### EXAMPLE 6

This Example describes that transfection of KIF14 siRNAs of different potencies results in more pronounced cytokinesis phenotypes in tumor cells than in normal cells.

Transgene overexpression, antibody microinjection and siRNA mediated gene silencing have been used to define the requisite roles of Rab6-KIFL, CHO1, and MPHOSPH1, three mammalian N6 kinesins that regulate cytokinesis (Hill et al. (2000) EMBO J. 19(21):5711-9; Matuliene et al. (2002) Mol. Biol. Cell. 13(6):1832-45; Abaza et al. (2003) J. Biol. Chem. 278(3)):27844-52). Phenotypes associated with the functional disruption of these gene products include induction of apoptosis, and/or formation of binucleate and multinucleate cells, all of which result from defects in cytokinesis (Hill et al. (2000) EMBO J. 19(21):5711-9; Matuliene et al. (2002) Mol. Biol. Cell. 13(6):1832-45; Abaza et al. (2003) J. Biol. Chem. 278(3)):27844-52). In this example, multiple siRNAs targeting KIF14 were used to investigate the effects of KIF14 depletion on cell division.

Cell culture and siRNA transfection: HCT116 and HeLa-S3 cells were cultured in DMEM supplemented with 10% fetal bovine serum (Gibco). Human renal epithelial cells (HRE) were obtained from Cambrex (East Rutherford N.J.). HRE cells were cultured in bullet kit (CC-3190) supplemented REGM™ according to Cambrex recommendations. All three cell lines were cultured at 37° C. in 5% CO₂. For siRNA transfections cells were seeded in 6-well (9.60 cm²) culture dishes at a density of 60,000-90,000 cells/well in 2 ml of an appropriate growth medium containing serum and incubated under normal growth conditions. Twenty-four hours after seeding, 100 pmols of siRNA was diluted in 70 microliters of serumfree OPTIMEM® and for complex formation, 5 microliters of Oligofectamine® (Invitrogen) transfection reagent diluted in 20 microliters of serumfree OPTIMEM was added to the diluted siRNA. After 20 minutes of incubation at room temperature, siRNA:Oligofectamine complexes were added drop-wise to cells directly. Cells were then incubated with the transfection complexes under their normal growth conditions until their collection for analysis at specific timepoints post transfection. All siRNA duplexes were purchased from Dharmacon (Lafayette CO.) Two KIF14 siRNA sequences and one KSP siRNA sequence were used. KIF14:204 (5′ AAACUGGGAGGCUACUUACTT 3′, SEQ ID NO:8), designated as weak, was selected using Tusch1 rules (AA leading dimer, >=75 bases downstream of the ATG, GC% range) and a specificity screen for FASTA hits. KIF14:3053 (5′ GUUGGCUAGAAUUGGGAAATT 3′, SEQ ID NO:23), designated as strong, was selected by a pseudorandom design algorithm which selects siRNAs evenly distributed across the gene in terms of GC%, base pair start and leading dimers. KSP:119 (5′ GGACAACUGCAGCUACUCUTT 3′, SEQ ID NO:24) was selected using oligoengine™ siRNA design software. Luciferase siRNA (5° CGUACGCGGAAUACUUCGAdTdT 3′, SEQ ID NO:10) was used as a control.

Quantitative PCR: Transfected cells were lysed in RLT buffer (Qiagen RNeasy Kit) containing 1% BME. Lysates were homogenized using QIAshredder spin columns (Qiagen) and total cellular RNA was purified using the RNeasy Mini kit (Qiagen). cDNA was synthesized from RNA using random primers and the reverse transcription reagent kit (Applied Biosystems). KIF14 and Glucuronidase beta (hGUS) mRNA expression was measured using Taqman real-time RT-PCR (SDS 7000 system, Applied Biosystems). Gene specific primer probes for KIF14 (Hs00208408) and hGUS (4310888E) were obtained from Applied Biosystems. Relative KIF14 expression was determined using the following calculation: relative expression =2^{ΔΔCt} where the ΔΔCt=(Ct_{target}−Ct_{hGUS})_{KIF14 siRNA}−(Ct_{target}−Ct_{hGUS})_{Luciferase siRNA}.

Western blot analysis of KIF14 expression: Transfected cells were trypsinized, collected by centrifugation (5 minutes at 300×g), and washed once with PBS. Cell pellets were resuspended in a small volume (<100 microliters) lysis buffer (20 mM Tris HCl pH 7.6, 150 mM NaCl, 1 mM EDTA, 1% Triton X-100, 1× protease inhibitor mix (Roche Complete®)) and incubated 10 minutes on ice. Following centrifugation (10 minutes at 10000×g), supernatant was collected and protein concentration determined using Bio-Rad DC Protein assay kit. 25 micrograms of each sample was subjected to SDS-PAGE on Bio-Rad Ready-Gels (7.5% acrylarnide or 4-15% acrylamide gradient). Proteins were transferred to a nitrocellulose membrane using a Bio-Rad Mini Trans-Blot® Transfer Cell according to manufacturer's instructions. Membrane was blocked in TBS-T buffer (150 mM NaCl, 10 mM Tris-HCl pH 7.6, 0.1% Tween-20) containing 5% non-fat dry milk (blocking buffer) for 30 minutes at room temperature with agitation. Membrane was then incubated with affinity-purified rabbit polyclonal anti-KIF14 (Abcam Inc. ab3746) diluted 1:1000 in blocking buffer for 90 minutes at room temperature with agitation. Membrane was washed 3 times in TBS-T then re-blocked. Membrane was incubated with HRP-conjugated goat anti-rabbit IgG (Zymed) diluted 1:10000 in blocking buffer for 45 minutes at room temperature with agitation. Following three washes in TBS-T, membrane was incubated in chemiluminescence detection reagents (ECL-plus, Amersham) and the image was captured using a CCD camera (Kodak Image Station 440CF).

Cell Cycle Analysis: To analyze cell cycle profiles, approximately 1×10⁵ to 5×10⁵ cells were harvested along with their accompanying media and pelleted by centrifugation in order to obtain both adherent and detached cells for subsequent flow cytometric analysis. Cells were resuspended in 200 microliters of PBS and fixed by addition of 1 ml of 100% cold ethanol on ice for 30 minutes. Cells were then pelleted and washed once with PBS to break up any clumps. Ethanol fixed cells were incubated at 37° C. for 30 minutes in PBS containing 10 micrograms/ml propidium iodide and 1 mg/ml RNaseA. For each sample, 10,000 events were collected using a FACSCalibur™ flow cytometer (Becton Dickinson) and incorporation of propidium iodide was used as a marker for DNA content. Cell cycle profiles were analyzed using FlowJo cytometry analysis software version 4.0.2.

BrdU incorporation Assay: HeLa-S3 and HRE were seeded separately in 96 well plates, (3×10³ cells/well), in 100 microliters of growth media. Twenty-four hours after seeding, cells were transfected with various siRNA oligos. Transfections were performed in accordance with the 6 well protocol described above. The amount of siRNA:oligofectamine complex added to 200 microliters media was adjusted to maintain a final siRNA concentration of 50 nM/well. Cellular proliferation was measured at 24, 48 and 72 hr post transfection using the colorimetric BrdU ELISA (Roche Applied Science). Cells were pulsed with 10 nM BrdU for 2.5 hr at 37° C. prior to cell fixation and DNA denaturation in accordance with the manufacturer's protocol (Roche). Fixed cells were incubated with 100 microliters of peroxidase-conjugated monoclonal anti-BrdU-POD antibody, diluted 1:100 (Roche), for 90 minutes at room temperature. Cells were then washed 3 times with 250 microliters 1×PBS and incubated with 100 microliters substrate solution (Roche) at room temperature until the appearance of a visible color difference was detectable between positive and negative controls. Light emission of each sample was measured at 370 nm (reference wavelength at 492 nm) using a spectrophotometer.

Results: Depletion of KIF14 induces tumor selective phenotypes that are associated with defects in cytokinesis and are a function of siRNA potency, as shown in Table 6. HeLa and HRE cells were transfected with four separate siRNA duplexes at 100 nM each (luciferase, KIF14:20, KIF14:3053, and KSP:119). Samples transfected with KIF14-specific siRNAs were analyzed 72 hours post transfection, and samples transfected with KSP-specific siRNA were analyzed 48 hours post transfection.

TABLE 6
Cell Cycle Profiles for siRNA-Transfected Cells

| Cells | siRNA | SubG1 (%) | Tetraploid (%) | Polyploid (%) |
|---|---|---|---|---|
| Hela | Luciferase | 3.19 | 15 | 3.2 |
| | KIF14: 204 | 49.5 | 11.5 | 1.67 |
| | KIF14: 3053 | 3.88 | 26.7 | 29 |
| | KSP: 119 | 43.5 | 30 | 5 |
| HRE | Luciferase | 3.26 | 13.4 | 1.08 |
| | KIF14: 204 | 11.4 | 12.9 | 1.25 |
| | KIF14: 3053 | 11.2 | 25.3 | 3.52 |
| | KSP: 119 | 2.14 | 57.3 | 1.52 |

Two specific phenotypes were observed in response to KIF14 silencing depending on the potency and endpoint efficacy of the particular siRNA. Weak siRNAs, such as KIF14:204, produced about 60 to about 80% KIF14 silencing and elicited apoptosis that was maximal at three days post transfection (Table 6) accompanied by an increase in binucleate cells. This phenotype is consistent with failure to complete cytokinesis after midbody formation (Abaza et al. (2003) J. Biol. Chem. 278(3):27844-52). Strong siRNAs, such as KIF:3053, produced more than about 80% KIF14 silencing and induced a marked accumulation of cells exhibiting tetraploid (4N) and polyploid (>4N) DNA content (Table 6) and multinucleate cells. Although evidence of cell death following transfection with strong KIF14 siRNAs was not seen in the shorter term experiment shown in Table 6, other experiments showed that such cells had a significant decrease in colony forming capacity. Continued chromosomal replication in the absence of cell division (endoreduplication) may occur in cells, such as HeLa, lacking a functional TP53- and RB1-regulated tetraploid checkpoint, which blocks the proliferation of cells that have entered G1 with a 4N DNA content (Hill et al. (2000) EMBO J. 19(21):5711-9). These polyploid cells would not be expected to persist in long term growth and would not form colonies. Therefore, the phenotypes elicited by both weak and strong KIF14 siRNAs indicate a role for KIF14 in cytokinesis.

Pronounced cytokinesis defects and/or apoptosis were also observed in other tumor cells following KIF14 depletion (SW480, HCT116 and A549). However, siRNA-mediated depletion of KIF14 in normal human renal epithelial cells (HRE) induced much more modest effects: there was an about 20% increase in binucleate cells and an about 50% reduction in overall cell growth after three days. Thus, KIF14 effects on cytokinesis were more pronounced in the tumor cells tested than in the normal cells. This tumor cell selectivity was not due to differences in KIF14 depletion, as silencing of KIF14 mRNA and protein were similar in both cell types. This selectivity was more pronounced for KIF14 depletion than for depletion of KSP (KIF11) (Table 6) or other mitotic kinesins with known roles in cytokinesis (KNSL5, RAB6-KIFL, and MPP1), spindle formation or chromosome movement (MCAK, CENPE).

The reason for tumor cell selectivity in KIF14 depletion is not currently understood. One plausible explanation from the literature is that most tumor cells lack the TP53/RB 1-regulated tetraploid checkpoint.

### EXAMPLE 7

This Example describes the expression and functional characterization of KIF14 motor domains.

Materials: Pfu polymerase and E. coli BL21 (DE3) was obtained from Stratagene. T4 DNA ligase, NdeI and XhoI were obtained from New England Biolabs. Ampicillin, carbenicillin was obtained from Sigma. pET22b was purchased from Novagen. E. coli TOP10 were from Invitrogen. MgCl₂, Tris-Cl, NaCl, imidazole, β-mercaptoethanol, lysozyme, PIPES, BSA, EGTA, and Na-ATP were purchased from Sigma. Tween was purchased from Aldrich, DTT was from Promega and KCl was from Fisher. Taxol® and tubulin (used to make microtubules) was purchased from Cytoskeleton. Quinaldine Red is from Acros.

K14 Motor Domain Cloning: The DNA sequence encoding a KIF14 motor domain (MD) spanning from V342 to K720 (SEQ ID NO:4) was amplified by Pfu polymerase in a PCR from a KIF14 cDNA (SEQ ID NO:1) cloned into a pBluescript plasmid vector. The primers used to amplify the DNA had flanking sequences that installed an NdeI site at the 5′ end and an XhoI site at the 3′ end (Primer 1: 5′-GTCTAGACATATGGTTCAGAACACCTCTGCA-3′, SEQ ID NO:11; Primer 2: 5′-TGCCTCGAGCTTCAATTCTCTAATTAACTT-3′, SEQ ID NO:12). An internal NdeI site was destroyed using a mutagenesis procedure known as Splicing by Overlap Extension (SOE). The resulting fragment was digested with the restriction endonucleases NdeI and XhoI and ligated to similarly treated pET22b plasmid vector. The ligation mixture was transformed into chemically competent E. coli TOP10 cells, cells selected for with ampicillin and desired clones screened for by Restriction Fragment Length Polymorphism (RFLP) and dideoxy nucleotide sequencing. A single positive clone was used as a PCR template with two additional primers (Primer 3: 5′-GTCTAGACATATGGTAGAGAATAGTCAAGTG-3′, SEQ ID NO:13; Primer 4: 5′-TGCCTCGAGATCTTCATTTACTTTAGCAAT-3′, SEQ ID NO:14) to generate DNA encoding three smaller MDs spanning from V342-D710 (SEQ ID NO:5), V354-K720 (SEQ ID NO:6), and V354-D710 (SEQ ID NO:7). All were digested, ligated and screened as was the original to generate single positive clones in the pET22b plasmid vector. The pET22b vector appends a DNA sequence to the gene that results in the expressed protein bearing 6 histidine residues at its C-terminus.

KIF14 Motor Domain Expression: All four clones were transformed into E. coli BL21 (DE3) cells and single colonies selected for gene expression. Cultures (0.5 L) were grown in Luria-Bertani medium supplemented with 2 mM MgCl₂ and 50 micrograms/mL carbenicillin at 18° C. for 50 hours after inoculation with a freshly saturated culture to 1% final volume. Cells were harvested by centrifugation and stored at −80° C.

KIF14 Motor Donmain Purification: All of the purification procedures were performed at 4° C. Cells were suspended in a lysis buffer (20 mM Tris-Cl pH 8.0, 300 mM NaCl, 0.1% Tween, 10 mM imidazole, 2 mM MgCl₂, and 5 mM β-mercaptoethanol) to which lysozyme was added to 1 mg/mL and allowed to react 10 min at 4° C. Cells were lysed in a Fisher Sonicator using a microtip with 4 pulses of 30 seconds each. Lysate was clarified by centrifugation at 60,000×g for 30 min and batch bound to Qiagen Nickel-NTA Superflow resin (bed volume 0.25 mL) for 120 min. Resin was collected by low-speed centrifugation and transferred to a Bio-Rad disposable column, where it was washed with 20 column volumes of lysis buffer. Protein was eluted from the resin with a step gradient of 5 column volumes of lysis buffer containing 20, 50, 100, 150 and 250 mM imidazole. Samples (10 microliters) of each fraction was analyzed on a 4-20% Tris-glycine SDS-PAGE gel (Novex). Fractions containing at least 50% of KIF14 MD (MW between 44.0 and 41.6 kDA) were pooled and dialyzed against 400 volumes of 20 mM Tris pH 8.0, 50 mM KCl, 2 MM MgCl₂, 01% Tween, 1 mM DTT and concentrated five-fold in a Centricon-30 (Amicon). Protein determination was according to the method of Bradford. Samples were divided into 5-6 aliquots, flash frozen in liquid N₂ and stored at −80° C.

KIF14 Motor Domain Assay: KIF14 MDs were assayed for microtubule (MT)-dependent ATP hydrolysis by measuring the rate of inorganic phosphate (Pi) release using the dye Quinaldine Red, which absorbs light of 540 nm when bound to Pi. Assays (50 microliters) contained 50 mM K-PIPES pH 6.9 (which contains 90 mM KCl), 1 mM EGTA pH 8.0, 1 mM DTT, 100 micrograms/mL BSA, 2 mM MgCl₂, 1 mM Na-ATP pH 7.0, 0.25-5 micromolar MT (which contain equimolar amounts of Taxol®), and 20-200 nM KIF14 MD enzyme. Reactions were initiated by the addition of enzyme and allowed to proceed at room temperature until they were quenched at regular time intervals by the addition of 50 microliters of 1.8 M KCl, 50 mM EDTA. To this was added 150 microliters of the Quinaldine Red dye solution (0.07 mg/mL quinaldine red, 0.09% polyvinyl alcohol, 4.1 mM ammonium molybdate, and 380 mM H₂SO₄). After 10 min incubation at room temperature, absorbance at 540 nM was read on a Molecular Devices Microtiter plate reader. Rates were calculated using the linear (steady-state) phase of the reaction.

Results: Table 7 shows the kinetic utilization of taxol-stabilized microtubules by partially purified KIF14 MD protein extracts prepared from E. coli cells expressing the four different KIF14 MD clones. k(obs) (min-1) refers to the rate of product formed (in micromoles/min) divided by the amount of enzyme (in micromoles). [Pi] released refers to the amount of product formed (in micromoles), and forms the numerator in the calculation of rate. All of the KIF14 MD proteins showed microtubule-dependent ATP hydrolysis activity. V342-K720 (SEQ ID NO:4) and V354-K720 (SEQ ID NO:6) displayed superior (and comparable) kinetic efficiency. These data demonstrate that the KIF14 MD protein, having sequence homology to other previously identified kinesins, such as Eg5 (Mayer et al. (1999) Science 286:971-974), has microtubuledependent ATP hydrolysis activity characteristic of other known kinesin proteins.

TABLE 7
Kinetic Utilization of Taxol-Stabilized
Microtubules by KIF14 MDs

| | V342-K720 | V342-D710 | V354-K720 | V354-D710 |
|---|---|---|---|---|
| | (SEQ ID | (SEQ ID | (SEQ ID | (SEQ ID |
| MT | NO: 4) | NO: 5) | NO: 6) | NO: 7) |
| [micro- | k(obs) | k(obs) | k(obs) | k(obs) |
| molar] | (min − 1) | (min − 1) | (min − 1) | (min − 1) |
| 0 | 0.00 | 0.00 | 0.00 | 0.00 |
| 0.25 | 0.36 | 0.03 | 0.73 | 0.39 |
| 0.5 | 1.57 | 0.73 | 1.49 | 1.07 |
| 1 | 2.79 | 0.79 | 4.12 | 1.87 |
| 2.5 | 9.10 | 3.02 | 9.29 | 5.79 |
| 5 | 13.25 | 5.91 | 18.30 | 10.18 |

A comparison of the kinetic characteristics of two of the KIF14 MDs with a panel of other human kinesin motor domains is shown in Table 8.

TABLE 8
Kinetic Characteristics of Human Kinesin Motor Domains

| | Catalytic | Kₘ (ATP) | K_{1/2} (MT) |
|---|---|---|---|
| Motor Domain | Rate | (micro- | (micro- |
| (amino acids) | (s⁻¹) | molar) | molar) |
| hu-KSP (1-367H) | 8.5 | 30 | 0.3 |
| (SEQ ID NO: 15) | | | |
| hu_KIF3A (1-350H) | 62.2 | 270 | 0.17 |
| (SEQ ID NO: 16) | | | |
| hu_uKHC (1-337H) | 14.9 | 1200 | 0.34 |
| (SEQ ID NO: 17) | | | |
| hu_nKHC (1-340H) | 2.6 | 900 | 1.1 |
| (SEQ ID NO: 18) | | | |
| hu_CENP-E (1-340H) | 6.8 | 240 | 1.6 |
| (SEQ ID NO: 19) | | | |
| hu_MKLP-1 (1-433H) | 1.5 | n.d. | 0.24 |
| (SEQ ID NO: 20) | | | |
| hu_KIF1B (1-350H) | 10.2 | 140 | 0.035 |
| (SEQ ID NO: 21) | | | |
| hu_KIF14 (342-720) | 1.1 | n.d. | 10.6 |
| (SEQ ID N0: 4) | | | |
| hu_KIF14 (354-720) | 0.235 | n.d. | n.d. |
| (SEQ ID NO: 6) | | | |

n.d. Not determined.

The kinetic parameters of the utilization of taxol-stabilized microtubules by KIF14 V342-K720 (SEQ ID NO:4) were obtained by fitting the data to the Michaelis-Menten equation, resulting in a kcat of 21.3+/−3.5 (s.d.) and a Km of 2.6+/−1.1 (s.d.).

### EXAMPLE 8

This Example describes the optimization of the efficiency of microtubule use of KIF14 motor domain v342-K720 (SEQ ID NO:4).

Two-Step Purification of KIF14 Motor Domain: The purification of the KIF14 motor domain v342-K720 (SEQ ID NO:4) up to the dialysis step was as described in EXAMPLE 7. Overnight dialysis was into cation exchange column buffer (50 mM HEPES pH 6.8, 1 mM MgCl, 1 mM EGTA, 1 mM DTT). Sample was applied onto an equilibrated 5 mL HiTrap SP HP, washed with 10 column volumes buffer, then eluted with a linear gradient to 750 mM KCl in buffer over 12 column volumes at a flowrate of 2 mL/min. Fractions (2 mL) were analyzed by SDS-PAGE (10%), and fractions with KIF14 MD purity of more than 90% were pooled, concentrated in a Centricon-30, and stored in 10% sucrose at −80° C. This procedure resulted in a yield of 0.4 mg/L of KIF14 V342-K720 motor domain (SEQ ID NO:4) with a purity of more than 95%.

Optimization of pH and Ionic Strength for KIF14 Motor Domain Activity: To optimize pH and ionic strength, the motor domain assay was performed as described in EXAMPLE 7, with the following modifications. For pH optimization, a series of 50 mM MES buffers spanning a pH range of 5.5 to 6.9, each with constant ionic strength, was used. For ionic strength optimization, the buffer was 50 mM MES pH 5.9 (containing 20 mM KCl) to which was added from 20 to 120 mM additional KCl. The pH optimum for the activity of the KIF14 V342-K720 MD protein (SEQ ID NO:4) was determined to be about 5.9. The optimal ionic strength of the buffer for KIF14 V342-K720 MD (SEQ ID NO:4) activity was found to be about 40 mM KCl.

Optimization of the pH and ionic strength resulted in increased efficiency of microtubule use by the KIF14 V342-K720 MD protein (SEQ ID NO:4). For example the kcat/Km for the V342-K720 motor domain (SEQ ID NO:4) under unoptimized conditions (K-PIPES, pH 6.7, 90 mM KCl) was 224, compared to 13.2 under optimized conditions (MES, pH 6.0,40 mM KCl).

Characterization of KIF14 Motor Domain Binding to Mg²⁺ and ATP: The motor domain assay was as described above, except that 50 mM MES pH 6.0, 20 mM added KCl was used, and either the MgCl₂ concentration was varied from 0 to 4 mM or the ATP concentration was varied from 0 to 1 mM. The optimal concentration of MgCl₂ for the activity of the KIF14 V342-K720 MD (SEQ ID NO:4) protein was found to be 1 mM. The minimum ATP concentration to achieve the maximal rate was determined to be 250 mM.

Effect of Temperature of KIF14 Motor Domain Activity: The motor domain assay was as described above, using 50 mM MES pH 6.0, 20 mM added KCl and varying the temperature was varied from 21° C. to 37° C. The rate of product formation by KIF14 V342-K720 MD (SEQ ID NO:4) was observed to increase 4.1 fold as temperature was increased from 21° C. to 37° C.

Suitability of K14 Motor Domains for high-throughput screening: The motor domain assay was as described above, using 50 mM MES pH 6.0, 20 mM added KCl, 0.5 micromolar MTs, a temperature of 37° C., and varying enzyme concentration from 0 to 10 nM. The signal to background ratio, calculated as the amount of Pi formed after a 90 minute incubation in the presence of enzyme relative to in the absence of enzyme, was determined using different concentrations of the V342-K720 motor domain (SEQ ID NO:4), and is shown in Table 9. A high enough signal over background was obtained at low enzyme concentrations to allow high-through-put screening (HTS). Moreover, the V342-K720 motor domain (SEQ ID NO:4) was stable in the reaction for at least 90 minutes.

TABLE 9
Signal to Background Ratio at Different
KIF14 Motor Domain concentrations

| Enzyme concentration (nM) | Signal to Background Ratio |
|---|---|
| 40 | 27 |
| 20 | 21 |
| 10 | 141 |
| 5.0 | 78 |
| 2.5 | 39 |

### EXAMPLE 9

This Example describes the identification of modulators of the activity of KIF14 motor domains.

Screen for KIF14 Modulators: The ATPase assay described in EXAMPLE 7, as optimized in EXAMPLE 8, was used to screen for compounds that modulated the activity of the KIF14 V342-K720 MD protein (SEQ ID NO:4). Some of the compounds tested were found to be candidate inhibitors of the KIF14 V342-K720 MD protein (SEQ ID NO:4), and four of these had selective inhibitory activities against the KIF14 MD compared to related kinesin motor domains KSP (SEQ ID NO:15), KIF3A (SEQ ID NO:16), uKHC (SEQ ID NO:17), nKHC (SEQ ID NO:18), CENP-E (SEQ ID NO:19), MKLP-1 (SEQ ID NO:20), KIF1B (SEQ ID NO:21), and MCAK (SEQ ID NO:22), as shown in Table 10. These four compounds were 1,1′-biphenyl-4-carbaldehyde thiosemicarbazone (compound 1), 4-isopropylbenzaldehyde thiosemicarbazone (compound 2; see, e.g., U.S. Pat. No. 3,849,575); 4-cyclohexylbenzaldehyde thiosemicarbazone (compound 3); and 4-isopropyl-3-nitrobenzaldehyde thiosemicarbazone (compound 4; see, e.g., Saripinar et al. (1996) Arzneimittel-Forschung 46(II):824-8).

TABLE 10
Characterization of 4 KIF14 MD Inhibitors

| | | | | | uKHC, nKHC, |
|---|---|---|---|---|---|
| | | | | | CENP-E, MKLP-1, |
| | | KIF14 | KIF3A | | KIF1B, MCAK |
| | | ATPase | ATPase | KSP ATPase | ATPase |
| IUPAC Name | Structure | IC50 (nM) | IC50 (nM) | IC50 (nM) | IC50 (nM) |
| 1,1′-biphenyl-4-carbaldehyde thiosemicarbazone (compound 1) | 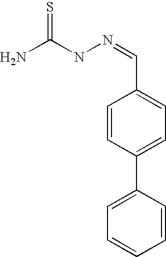 | 182 (n = 2) | 631 (n = 2) | >40000 (n = 2) | >40000 |
| 4-isopropylbenzaldehyde thiosemicarbazone (compound 2) | 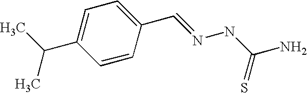 | 895 (n = 2) | 30982 (n = 2) | 25052 (n = 2) | >40000 |
| 4-cyclohexylbenzaldehyde thiosemicarbazone (compound 3) | 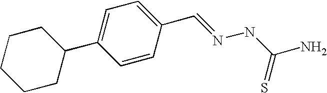 | 76 (n = 2) | 699 (n = 2) | 1529 (n = 2) | >40000 |
| 4-isopropyl-3-nitrobenzaldehyde thiosemicarbazone (compound 4) | 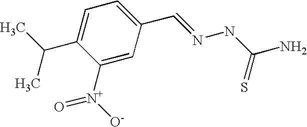 | 54 (n = 2) | 1550 (n = 2) | 1099 (n = 2) | >40000 |

Characterization of Candidate KIF14 Modulators in HeLa Cells: The effect of the 4 candidate KIF14 inhibitors identified in the screen (compounds 1-4) ere tested in HeLa cells using the alamarBlue™ assay for cell growth described in EXAMPLE 4. HeLa cells were plated at a density of 2000/cells per well in 10% DMEM in a 96-well plate (Costar, Cat. No. 3606) 20 hours prior to treatment with a KIF14 inhibitory compound in a series of dilutions (20 microliters of 11-fold concentrated solution of the compound added to 200 microliters of media). The cells were incubated at 37° C. for 72 hours prior to replacement of 100 microliters per well of the media with 10% (vol/vol) alamarBlue™ reagent. After incubation for 2 hours at 37° C., the plates were read on a spectrofluorimeter SpectroMax Gemini (excitation 544 nm, emission 590 nm). The background value (averaged from wells with no cells) was subtracted from each reading. The readings were normalized to 0% inhibition (or 100% viability) with the DMSO control and to 100% inhibition (or 0% viability) with a maximum inhibitory concentration of candidate KIF14 modulator. The three candidate KIF14 modulators tested exhibited an IC₅₀ between 1.0 and 5.0 micromolar, as shown in Table 11.

TABLE 11
Growth Inhibition in HeLa Cells by Candidate KIF14 Modulators

| [Compound] | | | |
|---|---|---|---|
| (nm) | Compound 1 | Compound 2 | Compound 3 |
| 19.5 | −3.58 | −5.39 | 8.97 |
| 39.1 | −4.74 | −8.04 | 5.97 |
| 78.1 | −2.50 | −5.77 | 8.16 |
| 156.2 | 5.29 | −8.75 | 6.91 |
| 312.5 | 10.32 | −7.21 | 8.88 |
| 625 | 12.76 | −5.51 | 12.32 |
| 1250 | 19.73 | −9.63 | 7.53 |
| 2500 | 51.68 | −2.57 | 44.36 |
| 5000 | 48.18 | 29.41 | 83.84 |
| 10000 | 40.98 | 61.19 | 95.02 |
| IC₅₀ | 1.0 micromolar | 5.0 micromolar | 2.8 micromolar |
| | (submaximal | (submaximal | |
| | inhibition) | inhibition) | |

Characterization of Candidate KIF14 Modulators in A2780 Cells: The effect of the four candidate KIF14 inhibitors identified in the screen (compounds 1-4) were tested in A2780 cells using the alamarBlue™ assay for cell growth described in EXAMPLE 4. A2780 cells were plated at a density of 4000/cells per well in RPMI1640 (containing 10% FBS, 1% Pen/Strep, 0.01 mg/ml insulin) in a 96-well plate (Costar, Cat. No. 3606) 16 hours prior to treatment. A compound dilution plate was prepared from a 10 mM stock solution using a 3-fold serial dilution series in DMSO. A 1.2 microliter aliquot of each concentration was transferred into 0.6 ml of medium. A 100 microliter aliquot of each concentration was added to the appropriate wells already containing 100 microliters of compound-free medium. After a 48 hour incubation at 37° C., 20 microliters of alamarBlue™ was added to each well (10% vol/vol). After an additional 6 hour incubation at 37° C., the plates were read on a spectrofluorimeter SpectroMax Gemini (excitation 544 nm, emission 590 nm). The background value (averaged from wells with no cells) was subtracted from each reading. The readings were normalized to 0% inhibition (or 100% viability) with the DMSO control and to 100% inhibition (or 0% viability) with a known control compound. The 4 candidate KIF14 modulators tested exhibited an EC₅₀ between more than 10000 and 3600 nM, as shown in Table 12.

TABLE 12
Growth Inhibition in A2780 Cells
by Candidate KIF14 Modulators

| [Compound] | | | | |
|---|---|---|---|---|
| (nM) | Compound 1 | Compound 2 | Compound 3 | Compound 4 |
| 0 | 5.0 | 5.0 | 5.1 | 1.5 |
| 13.7 | −4.5 | −1.0 | −5.3 | −7.0 |
| 14.1 | 11.6 | 14.6 | −8.1 | −4.0 |
| 123 | −10.3 | −18 | −10.4 | −9.2 |
| 370 | 19.0 | 9.4 | 7.8 | 4.4 |
| 1111 | 18.7 | 17.1 | 6.0 | 7.5 |
| 3333 | 23.6 | −2.6 | 39.8 | 25.0 |
| 10000 | 31.3 | 64.8 | 107.1 | 61.8 |
| EC₅₀ (nM) | >10000 | 8540 | 3600 | 7980 |

While the preferred embodiment of the invention has been illustrated and described, it will be appreciated that various changes can be made therein without departing from the spilit and scope of the invention.

1. A method for screening for modulators of a target protein, comprising the steps of contacting a target protein with a candidate agent and determining whether the candidate agent modulates the activity of the target protein, wherein the target protein comprises a sequence that has more than 80% amino acid sequence identity to the sequence provided in SEQ ID NO:2 or SEQ ID NO:3.

2. The method of claim 1, wherein (a) the target protein is contacted with the candidate agent at a first concentration and a first level of activity of the target protein is measured; and (b) the target protein is contacted with the candidate agent at a second concentration and a second level of activity of the target protein is measured, wherein a difference between the first level of activity and the second level of activity of the target protein indicates that the candidate agent modulates the activity of the target protein.

3. The method of claim 1, wherein the target protein is contacted with the candidate agent in vivo.

4. The method of claim 1, wherein the target protein is contacted with the candidate agent in vitro.

5. The method of claim 1, wherein a microtubule-stimulated ATPase assay is used for determining whether the candidate agent modulates the activity of the target protein.

6. The method of claim 1, wherein a binding assay is used for determining whether the candidate agent modulates the activity of the target protein.

7. The method of claim 6, wherein a microtubule-binding assay is used for determining whether the candidate agent modulates the activity of the target protein.

8. The method of claim 1, wherein a microtubule-gliding assay is used for determining whether the candidate agent modulates the activity of the target protein.

9. The method of claim 1, wherein a high throughput screening assay is used for determining whether the candidate agent modulates the activity of the target protein.

10. The methods of claim 1, wherein fluorescence, luminescence, radioactivity, or absorbance is used for determining whether the candidate agent modulates the activity of the target protein.

11. The method of claim 3, wherein contacting the target protein with the candidate agent in vivo comprises expressing the target protein in a cell.

12. The method of claim 3, wherein a cell viability assay is used for determining whether the candidate agent modulates the activity of the target protein.

13. The method of claim 3, wherein a cell morphology assay is used for determining whether the candidate agent modulates the activity of the target protein.

14. The method of claim 3, wherein a cell proliferation assay is used for determining whether the candidate agent modulates the activity of the target protein.

15. The method of claim 3, wherein a cell cycle distribution assay is used for determining whether the candidate agent modulates the activity of the target protein.

16. The method of claim 3, wherein an apoptosis assay is used for determining whether the candidate agent modulates the activity of the target protein.

17. The method of claim 1, wherein the target protein comprises the amino acid sequence provided in SEQ ID NO:2, SEQ ID NO:3, or a fragment of SEQ ID NO:3 having ATPase activity.

18. A method of modulating cell proliferation, comprising administering to a cell an effective amount of a modulator of the activity of a target protein, wherein the target protein comprises a sequence that has more than 80% sequence identity to the sequence provided in SEQ ID NO:2 or SEQ ID NO:3.

19. The method of claim 18, wherein the modulator is administered to a cell in vivo.

20. The method of claim 18, wherein the modulator is an inhibitor.

21. The method of claim 20, wherein the inhibitor is an RNA inhibitor.

22. The method of claim 21, wherein the inhibitor is a KIF14 RNA inhibitor.

23. The method of claim 22, wherein the KIF14 RNA inhibitor comprises the sequence provided in SEQ ID NO:8, SEQ ID NO:9, or SEQ ID NO:23.

24. The method of claim 20, wherein the inhibitor is a semicarbazone.

25. The method of claim 20, wherein the inhibitor is a thiosemicarbazone.

26. A method for treating a subject with a cellular hyperproliferation disorder, comprising administering to a subject with a cellular hyperproliferation disorder a therapeutically effective amount of an inhibitor of the activity of a target protein, wherein the target protein comprises a sequence that has more than 80% sequence identity to the sequence provided in SEQ ID NO:2 or SEQ ID NO:3.

27. The method of claim 26, where the cellular hyperproliferation disorder is cancer.

28. The method of claim 27, wherein the cancer is breast cancer.

29. The method of claim 26, wherein the modulator is an inhibitor.

30. The method of claim 29, wherein the inhibitor is an RNA inhibitor.

31. The method of claim 30, wherein the inhibitor is a KIF14 RNA inhibitor.

32. The method of claim 31, wherein the KIF14 RNA inhibitor comprises the sequence provided in SEQ ID NO:8. SEQ ID NO:9, or SEQ ID NO:23.

33. The method of claim 29, wherein the inhibitor is a semicarbazone.

34. The method of claim 29, wherein the inhibitor is a thiosemicarbazone.

35. A method for identifying candidate subjects for treatment with an inhibitor of the activity of a target protein, comprising the steps of:
(a) measuring the level of expression of a target protein in abnormally proliferating cells of a subject, wherein the target protein comprises a sequence that has more than 80% sequence identity to the sequence provided in SEQ ID NO:2 or SEQ ID NO:3; and
(b) identifying the subject as a candidate subject for treatment with an inhibitor of the activity of the target protein if the level of expression of the target protein in the abnormally proliferating cells is significantly higher than in control cells.

36. The method of claim 35, wherein the abnormally proliferating cells are breast cancer cells.

37. The method of claim 35, wherein the target protein comprises the amino acid sequence provided in SEQ ID NO:2, SEQ ID NO:3, or a fragment of SEQ ID NO:3 having ATPase activity.

38. The method of claim 35, wherein the level of expression of the target protein in abnormally proliferating cells is determined by measuring at the level of mRNA.

39. The method of claim 35 further comprising the step of treating the candidate subject with an inhibitor of the activity of the target protein.
